(19) United States
(12) Patent Application Publication
(10) Pub. No.: US 2003/0100043 A1
(43) Pub. Date: May 29, 2003

(54) Device and methods for automated specimen processing

(75) Inventors: Krishan L. Kalra, Danville, CA (US); Jason Zhang, San Jose, CA (US); Mitchell Lok, Hayward, CA (US)

Correspondence Address: CHARLES LOUIS THOEMING, 1990 N. CALIFORNIA BLVD., SUITE 720, WALNUT CREEK, CA, 94596, US

(73) Assignee: BioGenex Laboratories, Inc.

(21) Appl. No.: 09/816,849

(22) Filed: Mar. 23, 2001

Related U.S. Application Data
(60) Provisional application No. 60/191,988, filed on Mar. 24, 2000.

(51) Int. Cl. G01N 1/30; C12M 1/34

(52) U.S. Cl. 435/40.5; 435/287.3

(57) ABSTRACT

A device and methods for the automated histotechnological processing of a specimen are disclosed. The device comprises a microwave unit and a specimen positioning device which transports the specimen into a tank within the microwave unit where automated heating takes place. Preferably the microwave unit is controlled by a real-time microwave processor that permits control of the temperature within the microwave tank. Automated methods of dewaxing, antigen retrieval, nucleic acid retrieval, and hematoxylin and eosin staining which can be used in conjunction with the device are also provided. In one embodiment, a method is provided for simultaneous dewaxing and antigen retrieval; solutions useful for this method are also provided.

### TECHNICAL FIELD

This invention relates to a device, reagents and methods for automated histotechnological processing of pathologic specimens.

### BACKGROUND OF THE INVENTION

The human genome project and other genomic sequencing efforts have led to the identification of thousands of new genes encoding proteins of unknown function. The development of automated sequencing methods has enabled the revolution in sequencing which has resulted in the generation of huge amounts of sequence data.

One way in which functional information about new genes can be obtained is through characterization of the expression of the corresponding mRNAs and proteins in cells, tissues and whole organisms using in situ detection. Studying the effects of knocking out novel genes in an animal can also provide functional information; in situ detection methods are also used in characterizing the effects of such knockouts.

Traditionally, of course, the diagnosis of many diseases and disorders has employed in situ techniques. The reliability, accuracy, and reproducibility of the histotechnological method used is very important for proper diagnosis and treatment. In situ detection is useful for visualizing tissue morphology, disease markers, DNA and RNA and is helpful in complete and accurate prognosis and therapy selection.

Fixation of specimens is one of the most important methods allowing detailed study of the morphology and pathology of cells, tissues and organisms. Fixatives serve to stabilize, or “fix,” a specimen so that it maintains its integrity during subsequent processing. Fixatives also assist in visualization by bringing out differences in the refractive index of tissues. Fixatives can be categorized as coagulant or noncoagulant and additive or nonadditive based on their effects on the specimen. Some of the more widely used fixatives include the water-soluble alcohols and formaldehyde or paraformaldehyde. Different fixatives may be preferred for use in conjunction with particular histotechnological methods.

To obtain thin sections for microscopic study, fixed specimens are typically embedded with an embedding agent such as paraffin prior to sectioning. After the specimen is embedded and allowed to harden, it is sliced with a microtome to produce sections which are then affixed to a slide. However, embedding agents can interfere with staining, immunochemistry, and in situ hybridization. In order to perform subsequent procedures on the section, the embedding agent must first be removed in a process referred to as “deparaffinizing” or “dewaxing.” Techniques have been developed for dewaxing, typically involving organic solvents such as xylene. One recent advance has been the development of a dewaxing method which does not require the use of toxic organic solvents (Zhang et al., U.S. patent application Ser. No. 08/212,175 filed Mar. 11, 1994).

An unfortunate adverse effect of some fixation and embedding techniques is that antigenic epitopes in the specimen can be masked by these procedures. Techniques for “retrieving” the antigenicity of these epitopes have been developed and involve subjecting the specimen to various chemical or physical treatments. One technique useful for antigen retrieval in formaldehyde-fixed specimens is microwave heating of the specimen in solution (U.S. Pat. No. 5,244,787 issued Sep. 14, 1993 and assigned to BioGenex Laboratories, Inc.).

After fixation and dewaxing, the specimen can be stained with any of a number of well-characterized dyes following known protocols. Staining techniques serve to visually differentiate components of the tissue. Staining is often performed in conjunction with other visualization techniques such as in situ hybridization and immunochemistry; when used in this manner, staining can provide valuable information about where a given gene is expressed and where its encoded protein is localized and so help to reveal their function.

Staining techniques can be either progressive or regressive. In progressive staining, the sample is repeatedly exposed to the stain in discrete steps until the desired level of staining intensity is achieved. In regressive staining, the specimen is overstained and then treated with a differentiation, or decolorization, agent to produce the desired staining pattern. Regressive techniques are frequently used with mordant dyes.

Hematoxylin and eosin (H&E) staining is one of the most common methods performed in a pathology lab. Hematoxylin, upon oxidation, produces hematein, which is a weak anionic mordant dye that stains cell nuclei. Eosin is an anionic dye which combines with cationic tissue groups to produce different shades of pink in various components of the tissue. Proper H&E staining produces tissue sections having epithelial and muscle cell cytoplasm, collagen and erythrocytes of distinguishable shades of pink and clearly delineated blue cell nuclei. The staining pattern thus produced allows the histologist to identify the tissue and cell type and determine whether an abnormality exists.

Manual dewaxing, antigen retrieval and staining methods are tedious to perform and limit the number of samples that can be handled by a histotechnologist. Additionally, manual methods occupy valuable laboratory space, result in specimen-to-specimen variability, and require manual tracking of reagent use. Manual methods for microwave antigen retrieval are cumbersome and cannot be standardized. Manual antigen retrieval methods using commercially available microwaves demand constant monitoring to maintain the proper solution temperature without catastrophic boilover, which can contaminate the microwave and alter the composition of the solution. If sufficient fluid is lost from boiling in such methods, the specimen itself can be exposed and then directly heated by microwave radiation, which has been shown to adversely affect immunological staining. Excessive boiling can also subject the instrument and surrounding equipment to increased humidity, risking corrosion and short-circuits in electrical components.

There is a need in the art for automated methods designed to minimize human error, minimize human intervention, and to maximize reliability, robustness, and complete walkaway automation for performing histotechnological processes, and for devices and reagents useful in such methods.

### SUMMARY OF THE INVENTION

An apparatus and methods are provided for the automated histotechnological processing of a specimen. Reagents useful in performing such methods are also disclosed. The apparatus incorporates a microwave unit, preferably controlled by a real-time microprocessor, and a specimen positioning device for moving a specimen into and out of a tank located within the microwave unit. The apparatus thus can perform automated methods of dewaxing and/or antigen retrieval that require the use of elevated temperatures. Preferably the apparatus can also perform automated methods for staining specimens. Additional features can also be provided, including temperature sensors, a fluid delivery system, a filtered exhaust system, and a scanning device for sample identification. The apparatus and its components are controlled by a computer or other electronic control means.

Automation of antigen retrieval permits standardization of in situ detection processes and also decreases the risk of scalding, lowers the cost and increases the reliability of histotechnological methods, so that pathologic specimens of consistent quality can be obtained. Consistent specimen quality allows more reliable diagnosis and treatment and permits analysis of fewer specimens due to a decreased need for repetition. Additionally, greater numbers of samples can be processed by a given technician, who is also freed from performing tedious manual techniques using hazardous and toxic chemicals.

In one embodiment, an automated method is provided for simultaneously carrying out both dewaxing and antigen retrieval using the apparatus of the invention. Solutions useful for simultaneous dewaxing and antigen retrieval are also provided. This technique can also be applied to most tissues that have previously required special handling procedures such as enzymatic pre-treatment to obtain immunological staining.

### BRIEF DESCRIPTION OF THE DRAWINGS

FIG. 1 shows a schematic overview of a preferred embodiment of the apparatus. A loading deck is shown on the left of the figure where up to 12 specimen racks can be loaded. Four portable H&E tanks are shown at the bottom left where an automated method of H&E staining can be performed by the apparatus. MFB refers to the multifunction board.

Four microwave tanks (#1, #2, #3 and #4) are shown in the microwave processor chamber. The microwave tanks are supplied from a fluid delivery system that provides two to four different solutions to the microwave tanks. Containers holding the different fluids are pressurized using a common compressor; the pressure system includes a pressure gauge and an electronically operated pressure switch. The fluid supply lines are regulated by electronically operated valves under the control of a programmable computer system (not shown). The fluid delivery system for Solution #1 comprises a manifold that permits distribution of Solution #1 to any of the four microwave tanks. A common fluid outflow system drains the four microwave tanks using drainage pumps and a common pumping manifold; draining of the tanks is also controlled by electronically operated valves.

A door on the microwave chamber is electronically operated by a multifunction board in the programmable computer system; specimens are placed in the microwave tanks when the door is open, the door is shut, and the microwave is then operated to heat the solutions in the microwave tank(s). An interlock sensor on the microwave door detects the position of the microwave door.

Integrated holding and rinse tanks are shown on the upper right. Two holding chambers and a single rinse chamber are shown. The tanks are also supplied by the fluid delivery system and are drained by the fluid outflow system.

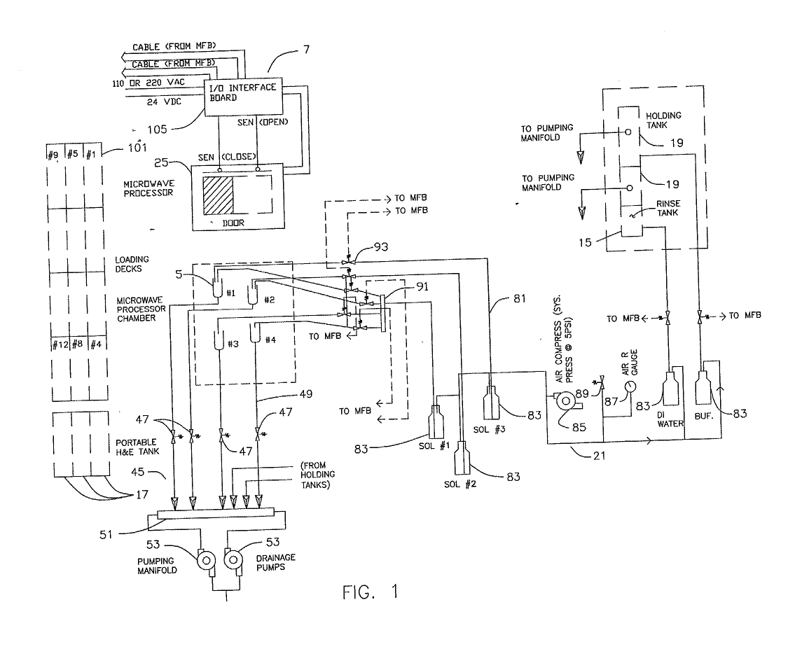

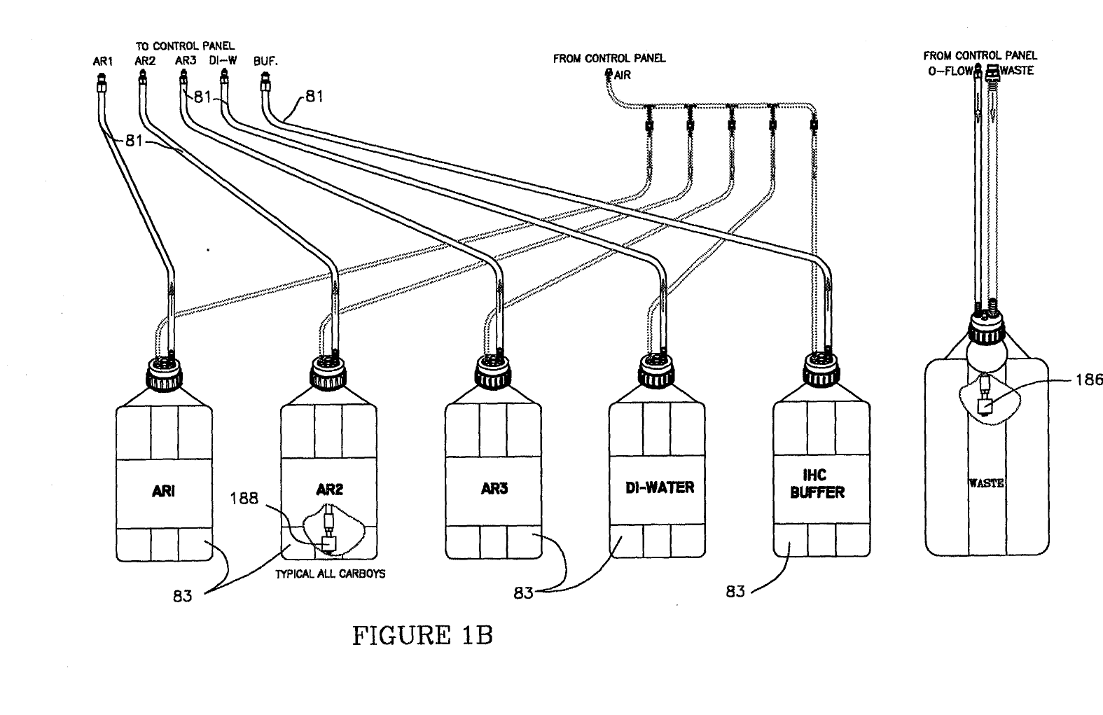

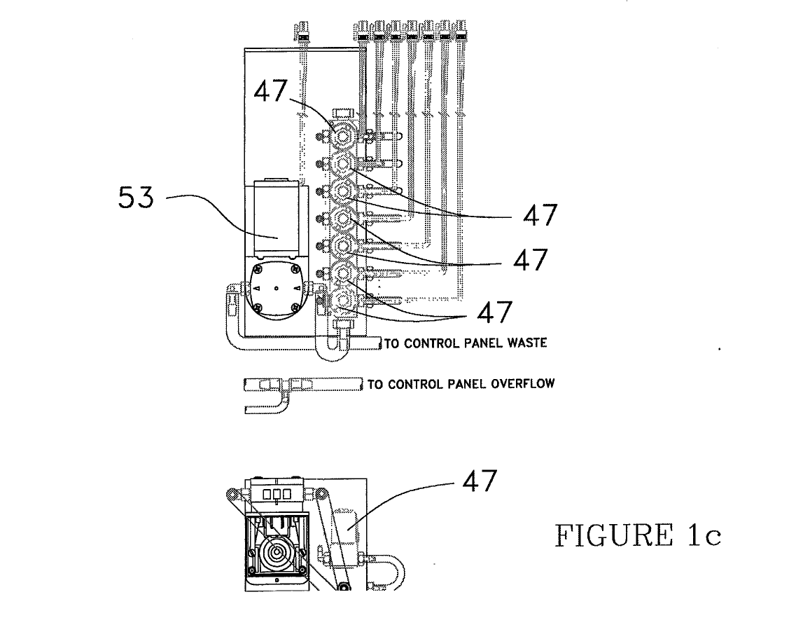

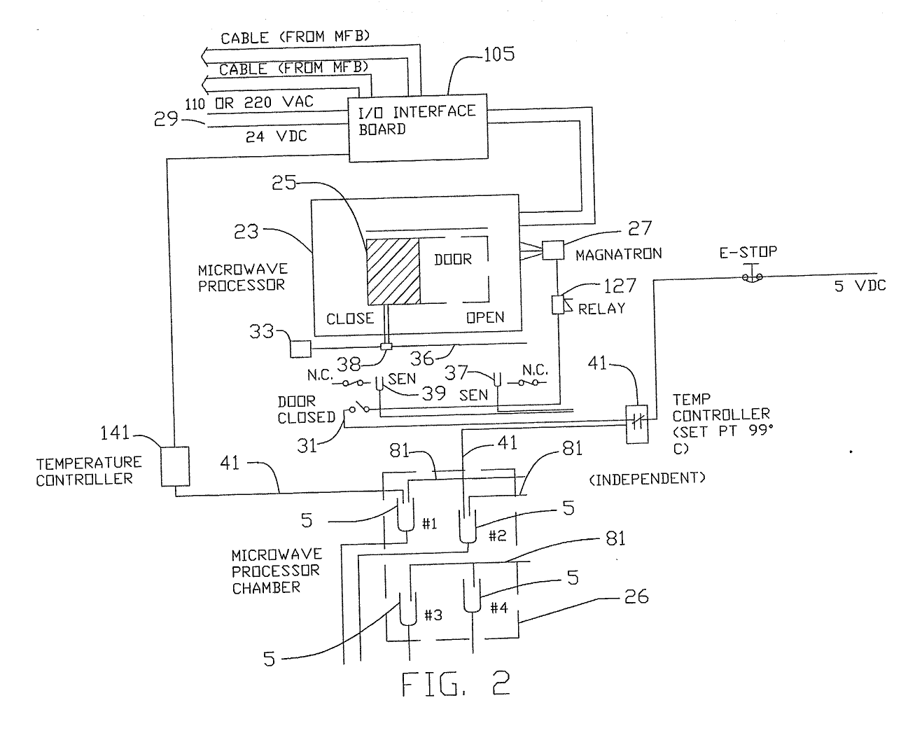

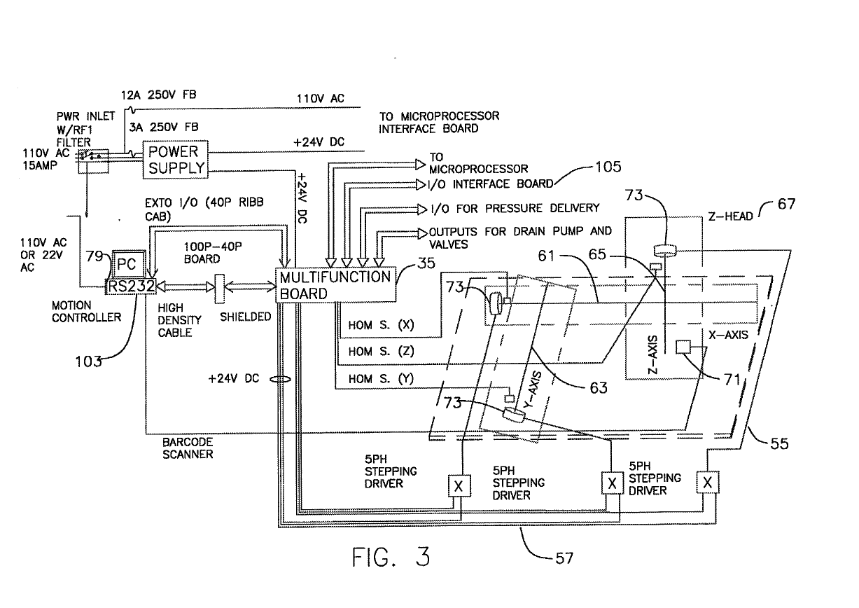

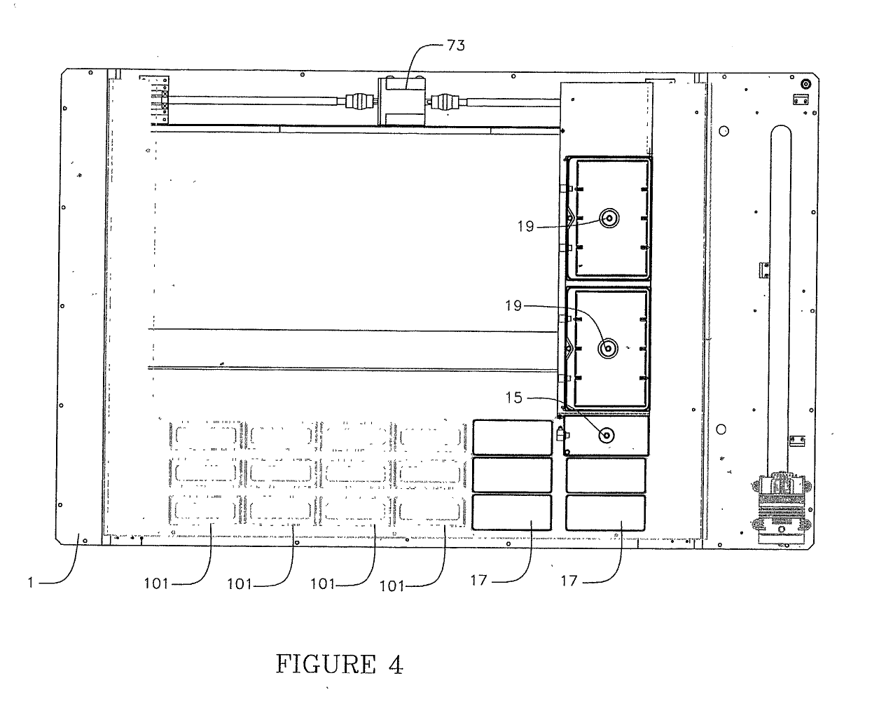

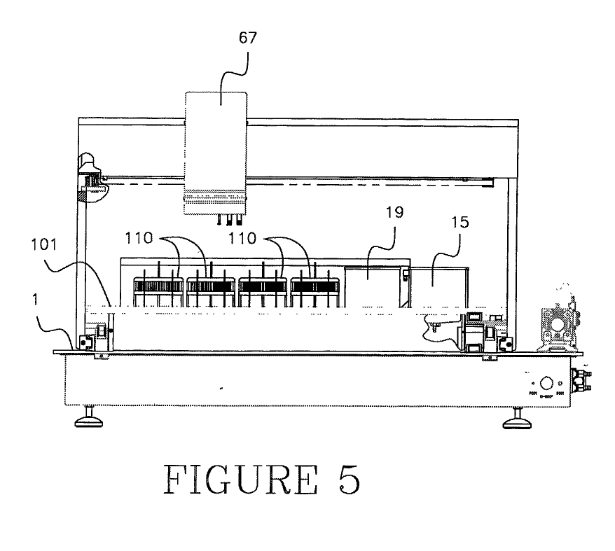

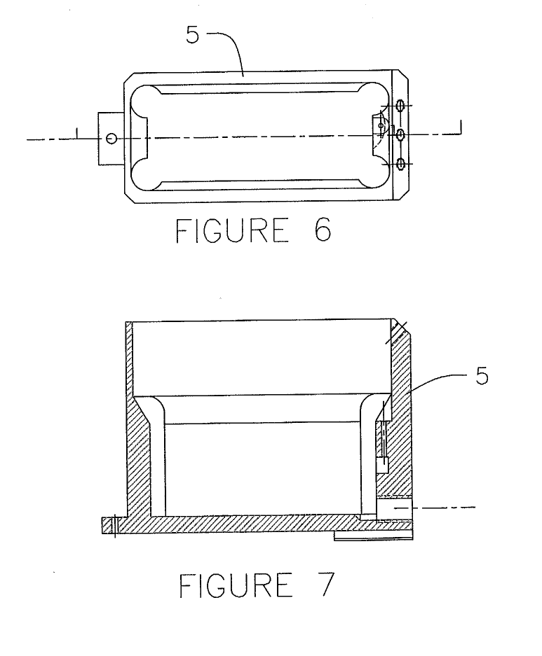

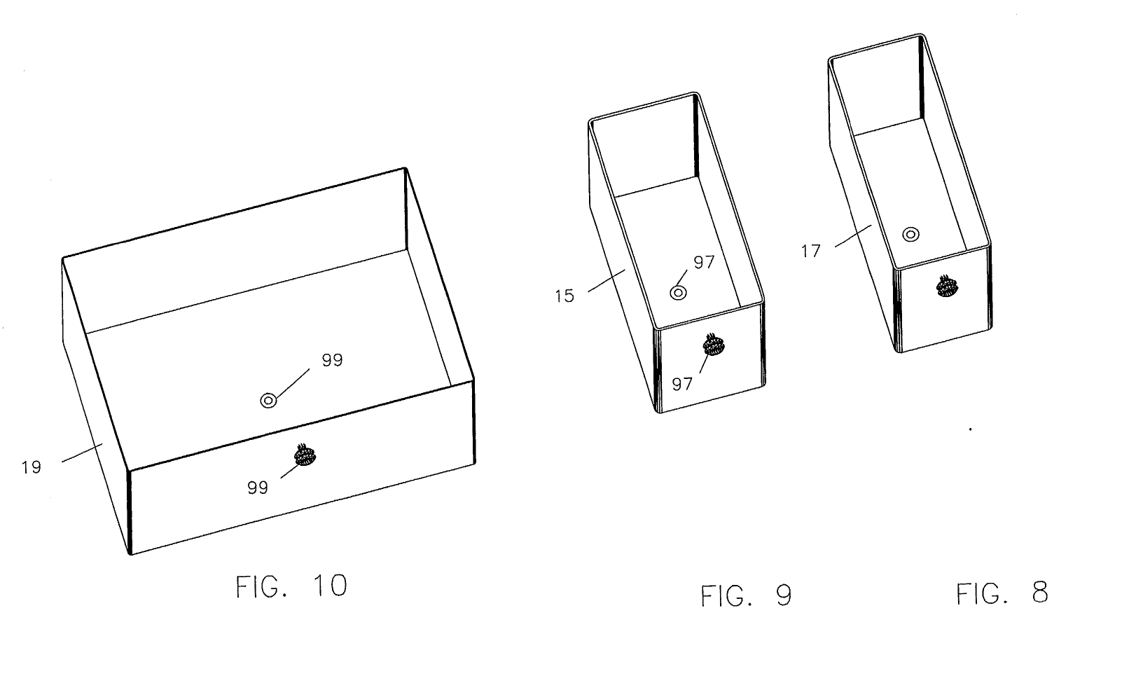

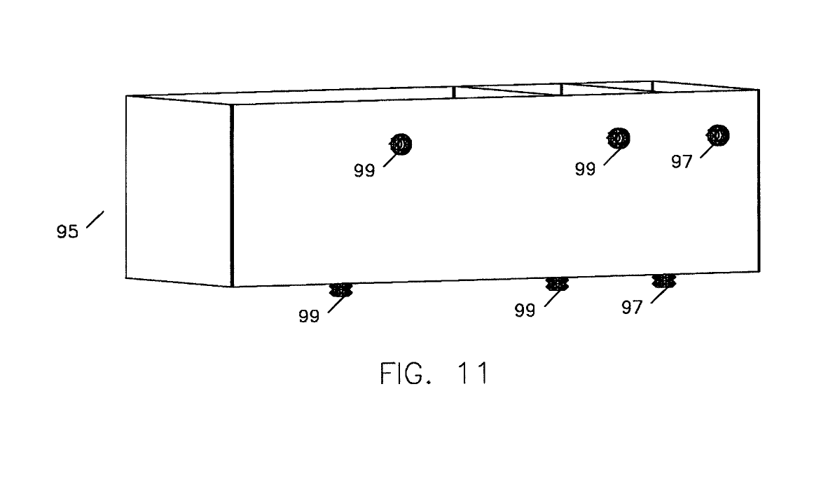

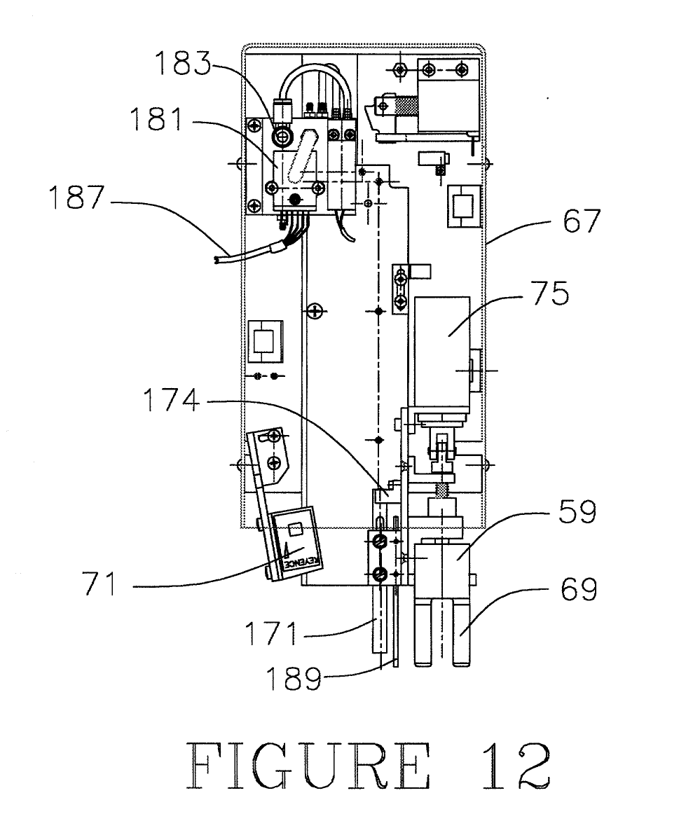

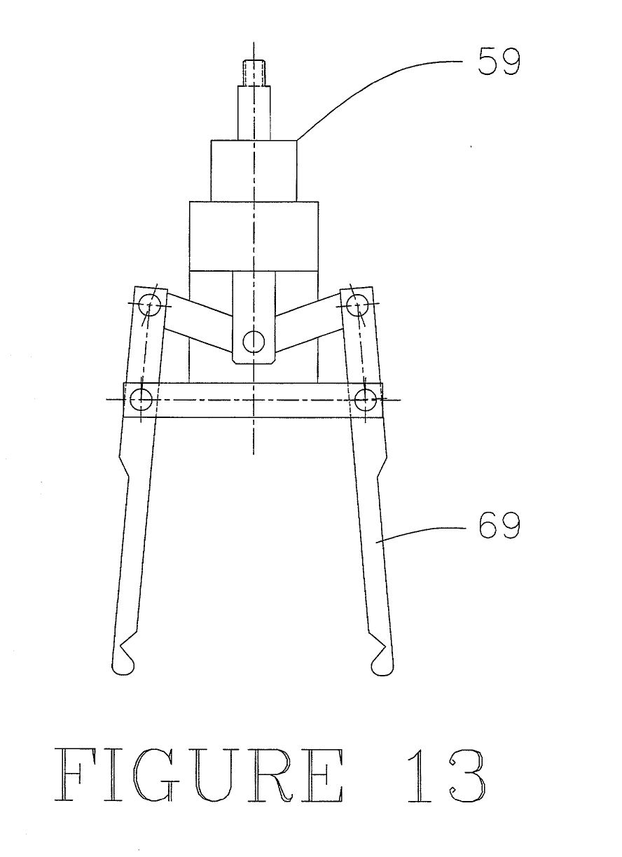

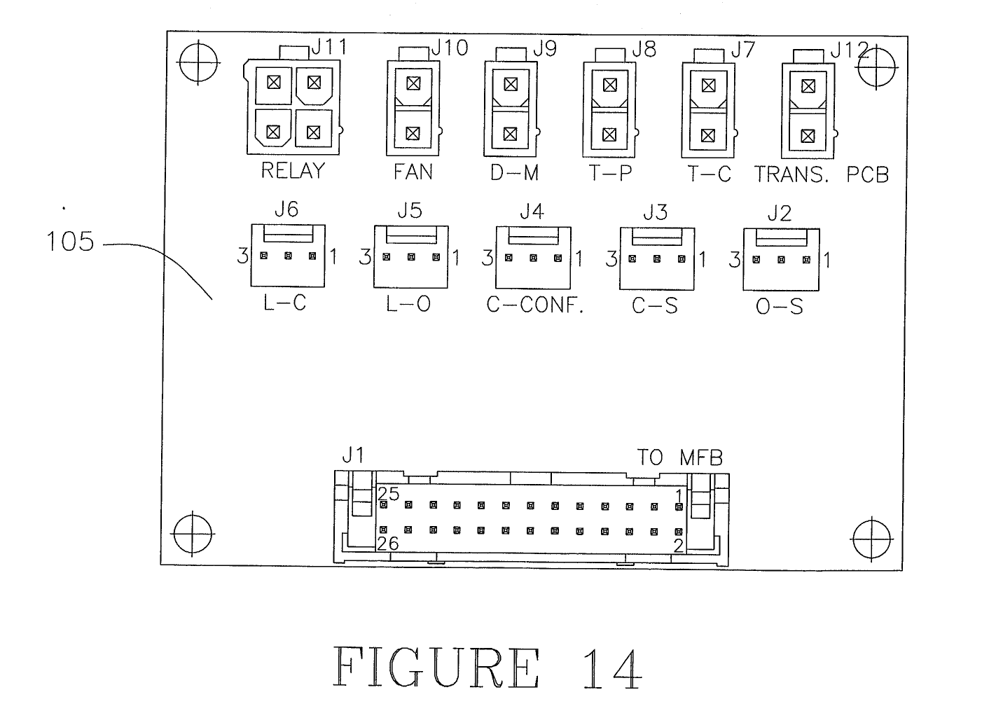

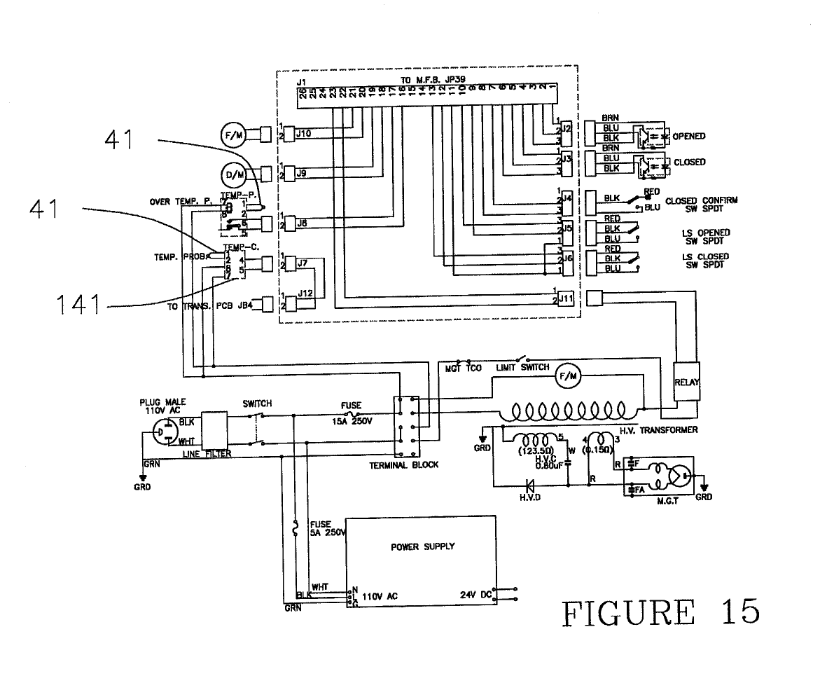

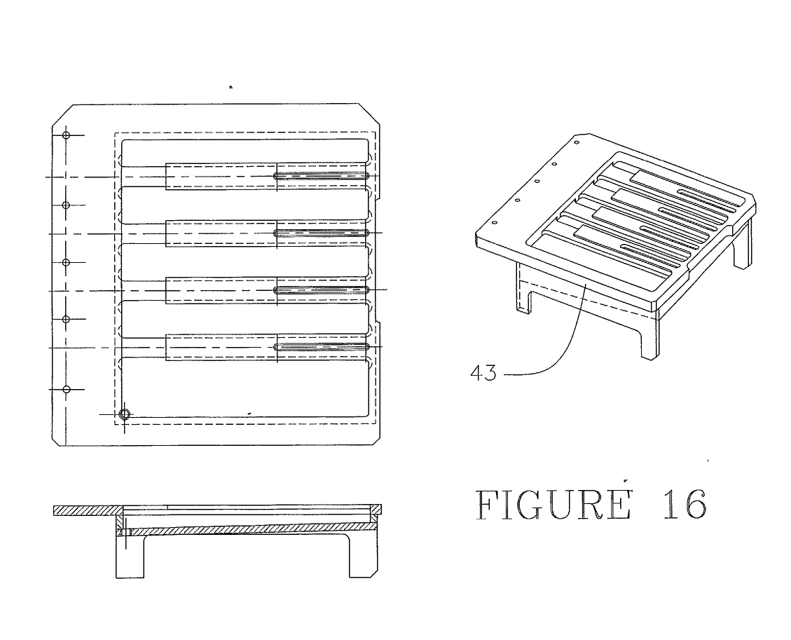

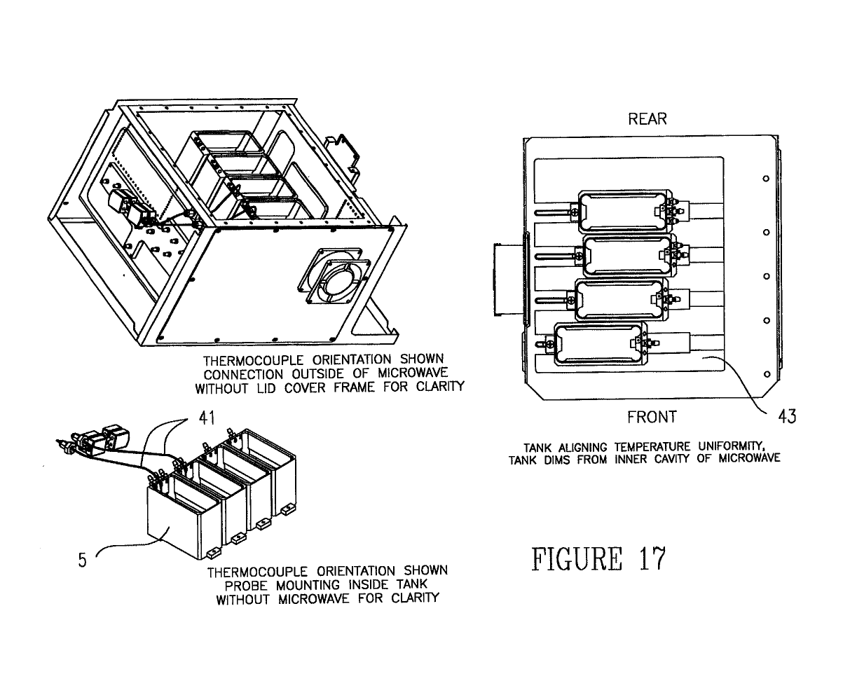

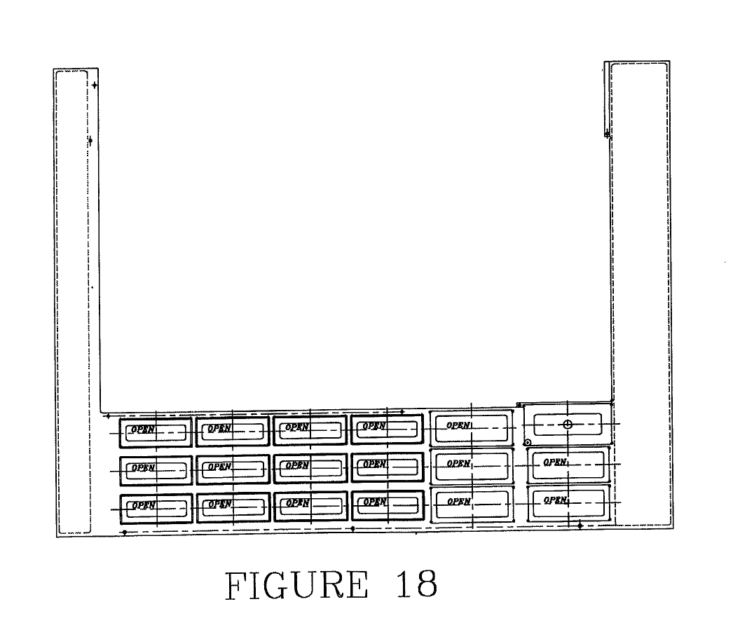

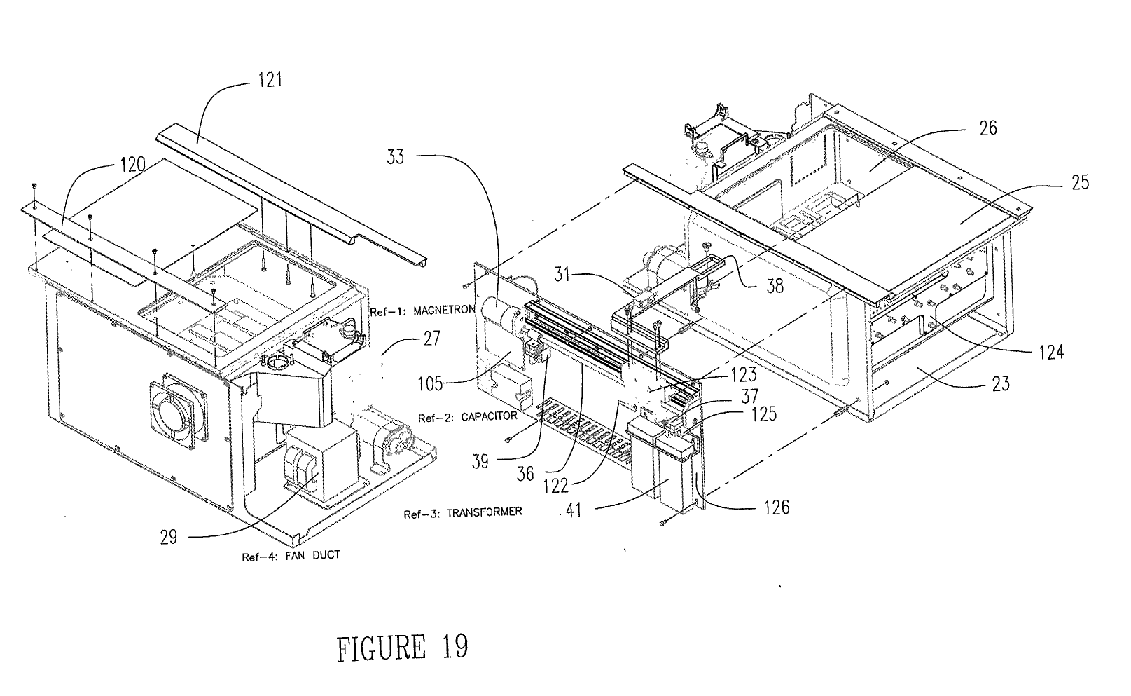

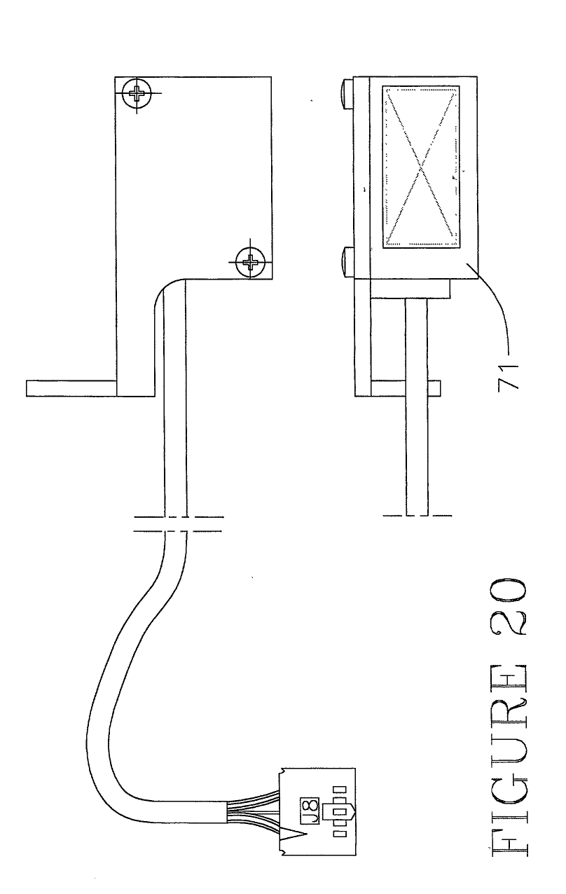

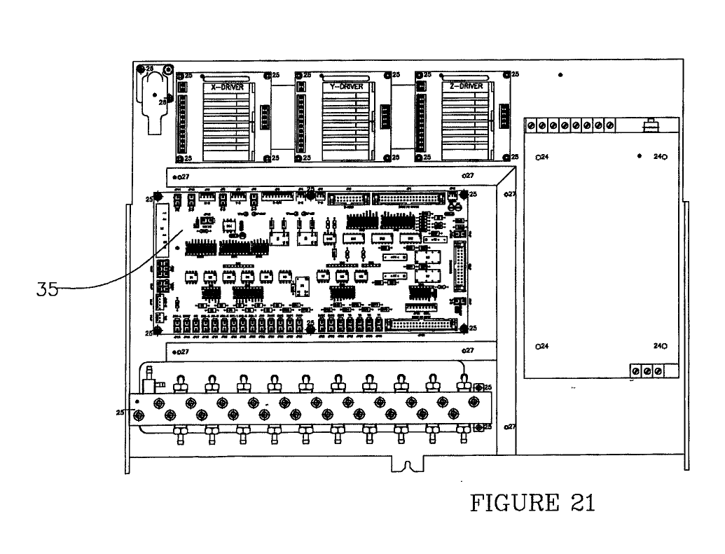

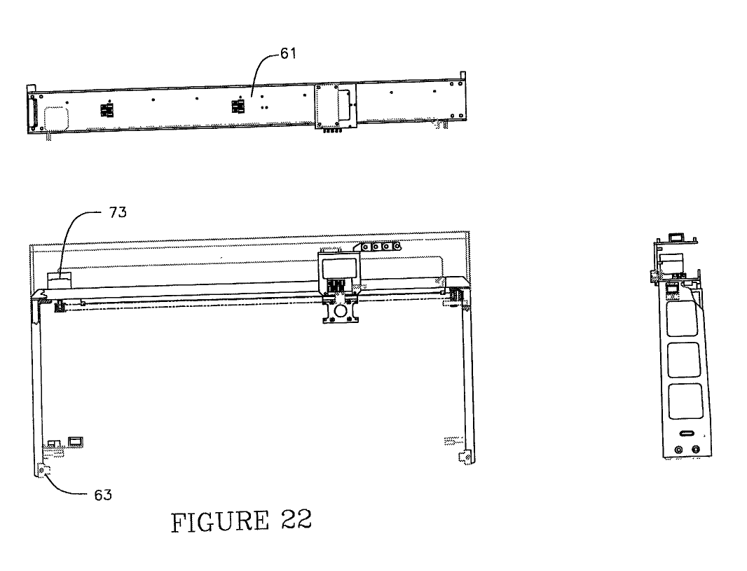

FIG. 1b shows the pressure delivery system.

FIG. 1c shows the fluid outflow system.

FIG. 2 shows a detail view of components of the microwave system. Control of the power supply to the magnetron that produces microwaves within the microwave chamber by the interlock sensor and a temperature controller that measures the temperature within a microwave tank is shown.

FIG. 3 shows a schematic view of the specimen positioning device, including the power supply circuitry, the motion controller and the programmable computer system that operates the specimen positioning device as well as the fluid delivery system and fluid outflow system.

FIG. 4 shows a top view of the apparatus, showing one arrangement of the loading deck, staining tanks and rinse tanks. The microwave system is not shown.

FIG. 5 shows a front view of the apparatus, showing four racks of specimen slides placed in the loading deck on the left, staining tank to the right of the loading deck, and a rinse tank on the right. The specimen positioning device is shown.

FIGS. 6 and 7 show top and profile views of a microwave tank used in the apparatus. The tank has design features in the corners which assist in the alignment of a specimen rack as it is lowered into the tank by the specimen positioning device. Connections for the fluid delivery and fluid outflow systems are shown.

FIGS. 8, 9 and 10 show embodiments of a staining tank, a rinse tank and a holding tank, respectively, that can be used in the apparatus. Fittings for attaching the fluid delivery and fluid outflow systems to the rinse and holding tanks are shown.

FIG. 11 shows an integrated rinse and holding tank. Two holding chambers are provided along with a single rinse chamber on the right. Fittings for independently supplying and draining each chamber are shown.

FIG. 12 shows a front view of the Z-head component of the specimen positioning device. The Z-head is moved along the Z-axis by a stepper motor linked to a gear engaged on a rail. The specimen holding device component of the specimen positioning device comprises a pair of gripper plates operated by solenoids. A barcode scanner is attached to the Z-head and detects identification labels on the specimen. In combination with the specimen positioning device and its associated motion controller, the barcode scanner allows assignment of positions to specimens within the apparatus.

FIG. 13 shows a detailed side view of the specimen holding device mounted on the Z-head, showing the pair of opposed gripper plates that are operated by solenoids to grasp and release a specimen rack.

FIG. 14 shows top view of a circuit board layout for a microwave processor I/O interface board used in conjunction with a programmable computer system that allows real-time regulation of the temperature of a solution in the microwave tank.

FIG. 15 shows a schematic of the circuit connections between the components of the interface board shown in FIG. 14. Connections to other components of the real-time microwave processor are shown outside the dashed lines.

FIG. 16 shows an inner base that can be used to position four microwave tanks within a modified microwave so that each tank maintains a similar temperature.

FIG. 17 shows the layout of the microwave tanks inside the microwave cavity. Thermocouples used to regulate the fluid temperature in the tanks are also shown.

FIG. 18 shows top views of the loading area.

FIG. 19 shows the components of one embodiment of the microwave chamber sealing means for placing a cover on the microwave chamber, including rails, front plate, flag mount, flag and fitting base and a switch mount.

FIG. 20 shows a bar code scanner that can be used in one embodiment of the specimen identification device.

FIG. 21 shows an electrical assembly for controlling the operation of the system.

FIG. 22 shows an assembly gantry forming part of one embodiment of the specimen positioning device.

### DETAILED DESCRIPTION OF THE INVENTION

An automated method and apparatus for histotechnological processing of a specimen is disclosed. The apparatus includes a specimen positioning device which transports the sample in three dimensions within the apparatus. The apparatus includes a microwave unit containing a microwave tank into which the sample can be placed by the specimen positioning device. A solution is provided in the tank which permits the sample to be dewaxed or to have its antigens retrieved, or both, upon microwave heating. Compositions useful in such methods are also provided. The apparatus preferably also can automatically stain the specimen.

Before the present invention is described in detail, it is to be understood that this invention is not limited to the particular methodology, devices, solutions or apparatuses described, as such methods, devices, solutions or apparatuses can, of course, vary. It is also to be understood that the terminology used herein is for the purpose of describing particular embodiments only, and is not intended to limit the scope of the present invention.

Use of the singular forms “a,” “an,” and “the” include plural references unless the context clearly dictates otherwise. Thus, for example, reference to “a microwave tank” includes a plurality of microwave tanks, reference to “a fluid source” includes a plurality of such fluid sources, reference to “a specimen position device” includes a plurality of specimen positioning devices, and the like.

As used herein, terms such as “connected” and “attached” encompass direct or indirect connection or attachment, unless context dictates otherwise. Where a range of values is recited, it is to be understood that each intervening value, to the tenth of the unit of the lower limit of that range, between the recited upper and lower limits of that range is also specifically disclosed, unless the context clearly dictates otherwise. Each smaller range between any recited value or intervening value in a recited range and any other recited or intervening value in that recited range is encompassed within the invention. The upper and lower limits of these smaller ranges can independently be included in or excluded from the range, and each range where either, neither or both limits are included in the smaller range is also encompassed within the invention. Where the recited range includes one or both of the limits, ranges excluding either or both of those included limits are also within the scope of the invention. Where the value being discussed has inherent limits, for example where a component can be present at a concentration of from 0 to 100%, or where the pH of an aqueous solution can range from 1 to 14, those inherent limits as well as any intervening value between an inherent limit and any recited value are specifically disclosed, along with ranges defined by any such value or limit, as described above. Where a value is explicitly recited, it is to be understood that values which are about the same quantity or amount as the recited value are also within the scope of the invention.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

The Specimen

The specimen is typically a pathologic sample. The specimen can be a section of tissue obtained, for example, by surgery or autopsy and processed using histotechnological techniques, or the specimen can be a biological specimen such as an aspirate obtained from the lung, a needle biopsy, or a biological fluid such as sputum. The specimen can be obtained from an animal of any species or from deposits left by such an animal. The specimen can be obtained, for example, from a human or other primate, a mammal, a domesticated animal, for example a cow, horse, goat, pig, llama, alpaca, rabbit, sheep, dog, cat or ferret, a commercially-raised animal, for example an ostrich, buffalo, deer, a pet, for example a fish, bird, reptile, or amphibian, or any animal treated by a veterinarian. The specimen can be obtained in a medical or veterinary setting, or can be obtained in the wild.

The specimen is typically treated with a fixative and embedded in paraffin medium and then cut into thin sections prior to performing a method of the invention. The choice of fixative can be influenced by the specimen type and the particular component to be visualized. Determination of the fixative to be used is within the skill of the art. The fixative used can also determine the need for particular methods of the invention to be performed. For example, a specimen fixed by any fixing process that uses formalin (or a different formaldehyde derivative or form) as a tissue-fixing agent may advantageously be subjected to automated antigen retrieval.

A specimen embedded in paraffin can be subjected to manual or automated dewaxing, for example as described below. Although the invention provides an automated method of simultaneous dewaxing and antigen retrieval, it is possible that the specimen could be dewaxed first by another method and then subjected to an antigen retrieval or staining method of the invention. Similarly, the specimen could be dewaxed by this system and retrieved or just stained.

Where the specimen is a tissue, the specimen is typically sectioned after fixation and embedding. The thickness of the section is chosen to permit acceptable visualization of the specimen with the techniques used.

Apparatus of the Invention

The apparatus of the invention comprises a supporting framework 1, a microwave 23, a microwave tank 5 located within the microwave, microwave source control means 7, a specimen positioning device 55 attached to the framework, and specimen positioning device control means 11. Preferably, the apparatus also can comprise, individually or in any combination, a specimen identification device 71, a rinse tank 15, staining tanks 17, a holding tank 19, and a fluid delivery system 21, along with other components described below.

Supporting Framework

The supporting framework 1 serves to integrate the various components of the apparatus and to protect the user from the machinery, solutions and fumes during operation. The framework provides a stable structure for attaching the microwave unit and the specimen positioning device, and permits the movement of the specimen positioning device within the framework to different work areas of the apparatus. The particular architecture of the supporting framework is not critical. In one embodiment, the supporting framework 1 (the bottom of which is shown in FIG. 4) is a cabinet constructed out of steel. The cabinet is a one-piece housing comprised of a cover and a body. One or more of the panels, or the entire upper portion of the cabinet, can be formed as an integrated assembly. The cabinet is desirably formed to permit easy assembly of the apparatus and easy access to the interior so that specimens can be loaded and removed. Preferably, the cabinet is sealable so that fumes and odors arising during the operation of the apparatus can be controlled. The interior space enclosed by the cabinet can be accessed through a door or access port on one side of the cabinet, or the side or top of the cabinet may open, for example on hinges. Any combination of access ports, doors, windows, cutouts, or other openings can be present in the cabinet. However, the cabinet is preferably sealed while the apparatus is in operation; a sensor can be included in the apparatus which detects whether the cabinet is closed and halts operation of the apparatus when it is not. A system for exchanging the atmosphere in the cabinet can be incorporated. Preferably, a fan is attached to the cabinet so that the internal atmosphere can be forced out. The atmosphere exiting the chamber is preferably filtered prior to egress to minimize the release of undesired molecules, for example by including a charcoal filter or series of filters which filters the atmosphere being removed from the cabinet.

The Microwave

The microwave for heating the specimen comprises of a microwave unit 23, microwave chamber sealing means 25, and microwave source control means 7. The microwave unit comprises a sealable microwave chamber 26, a microwave source 27 directing microwaves into the chamber, and power supply means 29 for the microwave source. The microwave preferably also comprises an interlock sensor 31 that delivers a signal when the microwave chamber is not sealed which prevents the production of microwaves by the microwave source (FIG. 2).

The chamber can be any chamber suitable for containing microwave radiation, examples of which are known in the art. The chamber includes a sealable access port or opening on at least one side so that the chamber can be opened for manipulation of specimens within the chamber and sealed during microwave heating. The microwave chamber sealing means 25 can be a hinged or sliding door, wall or top of the microwave, or a cover that be positioned over the access port or opening of the chamber. Preferably the chamber comprises a microwave-impermeant door. The door can be attached to the chamber in any suitable manner, for example by hinges or rails 120, 121. Preferably, the door seals the top of the microwave chamber so that, when open, it allows convenient access to the microwave tanks from above by the specimen positioning device.

The opening and closing of the door to unseal and seal the microwave chamber can be controlled by any electronically-operated mechanism for pulling, pushing or placing the seal onto the microwave chamber. In a preferred one embodiment, the microwave-impermeant door is opened and closed by an electric motor 33 controlled by a multifunction board 35 in a programmable computer system. The motor is preferably a DC motor which can be driven in either direction by inverting the current, which can be done by the multifunction board. The DC motor drives a lead screw 36 which is engaged with a door slide 38 on the microwave door. A door open sensor 37 and a door closed sensor 39 are preferably incorporated to permit detection of the position of the door. While performing an operating sequence where the chamber is to be opened, for example while cooling the chamber or when access to the microwave tanks by the specimen positioning device and/or specimen identification device is required, the computer drives the opening of the door until the open door sensor is triggered. When the operating sequence calls for the chamber to be closed, for example when the microwave is to be operated, the computer drives the motor in the closing direction until the door open sensor signals that the door is not open and the door closed sensor signals that the door is fully closed. The microwave source can then be operated to produce microwaves in the microwave chamber.

The microwave source can be any microwave oscillator that can provide microwaves of sufficient energy and frequency to heat the specimen in solution in the microwave tank to dewax the specimen or allow for retrieval of antigenic detectability of components of the specimen. Suitable microwave sources are known in the art. A preferred microwave source is a magnetron. Preferably the microwave source can supply at least about 300 watts of power, and typically will provide from about 300 to about 2,000 watts of power, more preferably from about 500 to about 1,000 watts of power. The microwave source should provide a frequency in the range of from about 1 to about 50 GHZ; commercially available sources include magnetrons which typically provide a frequency of 2.45 GHZ. The microwave source is typically attached on the outside of the microwave chamber within a waveguide which directs the microwaves produced by the source into the chamber.

Any power source that can supply sufficient power to operate the microwave can be used, for example a power grid provided by a utility company having an outlet, a generator, or a battery. The microwave power supply means can comprise any arrangement of cords, receptacles, transformers, inverters and other circuitry that can direct power of different voltages and of alternating or direct current from the power source to the microwave source under the control of the microwave source control means. Examples of suitable microwave power supply means are known in the art. Preferably, the microwave power supply means operates on 110 volts alternating current (VAC) or 220 VAC, and more preferably can operate at either voltage.

The microwave source control means can be any device which can direct the initiation and termination of microwave production by the microwave source. In one embodiment, the microwave source control means comprises a programmable computer system electrically operably connected to the microwave source. Preferably, the microwave source control means incorporates a real-time microwave processor comprising a transformer and one or more thermal sensors, for example a thermocouple. The real-time microwave processor can detect a signal from a temperature sensor, for example as shown in FIGS. 2 and 17 a thermocouple 41, indicating the temperature in a solution in the microwave tank. This signal is sent though a temperature controller 141 to the computer where it is compared to the setpoint temperature by the software and depending on the difference a command is sent out to control the solid state relay 127 and the output of the microwave. This methodology is used to maintain the temperature of the solution.

Where two or more thermal sensors are used, upper and lower sensors can be set to allow control of the microwave tank solution temperature within a narrow range.

Preferably the microwave source control means can maintain the temperature of the solution in the microwave tank in the range of from at least about 45° C. to about 130° C. More preferably, the solution temperature is maintained at or above about 65° C., and most preferably at or above about 98° C. Where the solution is aqueous or predominately aqueous, the temperature is preferably maintained in the range of about 95° C. to about 99° C., and most preferably from about 98° C. to about 99° C. Other solutions may, of course, have higher boiling points which permit the use of a higher temperature.

In one embodiment, the microwave is adapted from General Electric model number JE740WY, which provides about 700 watts of power. The microwave is adapted by removing the top from the instrument and replacing it with a sealable door 25 whose operation is controlled by the multifunction board 35, as shown in FIGS. 1 and 2. Two thermocouples are used to measure the temperature of solutions in the microwave tanks; one is used as an over-temperature cutoff and is set at 99° C., the other is used as a control to signal the production of microwaves when the solution is below a set temperature, which is preferably about 98° C. Both thermocouples send signals to external controllers mounted on the outside of the microwave chamber. Bulkhead fittings are added to allow solutions to pass into and out of the microwave chamber for filling and draining the microwave tanks. Where a plurality of tanks are provided within the microwave, they are preferably arranged so that each tank receives a comparable amount of microwaves and thereby maintains a solution temperature approximately the same as the other tanks within the chamber. Determining suitable locations and orientations within the microwave that result in such solution temperatures within the microwave tanks is within the skill of the art. Referring to FIG. 16, an inner base 43 can be provided which positions four microwave tanks within a modified GE JE740WY microwave so that each tank maintains a similar temperature. This would be the first procedure performed to tune up the system before any processing can be commenced.

Another way of controlling temperature in the tanks without turning the microwave power on or off may be by providing a baffle at the end of the microwave cavity. The baffle will be mounted on a shaft controlled by a motor. The baffle could be made of a microwave absorbing or reflecting material. The energy absorbed or reflected by the baffle could be influenced by the baffle area exposed to the microwaves. This exposed are could further be controlled by rotating the baffle connected to the motor.

So in a control scheme, the thermocouple signal would be read and compared with the target temperature. Depending on the difference in temperature, a signal would be sent to rotate the motor to being the baffle at a determined angular position and, thus, make more or less energy available for the tank fluid heating.

Microwave Tank

The microwave tank can be made of any material suitable for use at elevated temperatures with the solutions described herein and compatible with microwave radiation when the tank is filled. The microwave tank is located within the microwave chamber so as to be accessible to the specimen positioning device. A plurality of microwave tanks can be provided within the microwave chamber; if so, they are preferably made of a material that is compatible with microwave radiation when the tank is not filled with solution, for example teflon or glass, so that not all the tanks need to be filled with solution for each run.

The microwave tank must be large enough to contain at least one specimen in a solution, and preferably is large enough to hold a plurality of specimens. In a preferred embodiment, the microwave tank is large enough to hold at least one rack of the type commonly used in histotechnological methods for holding a plurality of slides parallel in a vertical position and which can be obtained from laboratory supply distributors such as VWR and Fisher. Where a rack is to be used, the microwave tank preferably has a flat base so that the rack, which has a flat bottom, will sit levelly within the tank. This allows the rack to be reliably located by the specimen positioning device. Although the microwave tank could conceivably fill the entire microwave chamber, it is preferably only slightly larger than the specimen or rack that it is designed to hold, in order to minimize the volume of solution needed to fill the tank and therefore minimize the amount of time required to heat the solution.

One embodiment of the microwave tank 5 is shown in FIGS. 6 and 7. In this embodiment, the tank has internally sloped slides that guide a specimen rack into the correct position within the tank as it is lowered by the specimen positioning device. The internal corners at the top of the tank are rounded so as to accommodate some deviation in orientation as the rack is initially lowered (FIG. 6). The tank can be manufactured to specification from teflon.

Preferably, the microwave tank has an outflow system 45 for conducting fluid out of the microwave chamber as shown in FIG. 1. Fluid outflow is preferably regulated by an outflow valves 47 and outflow valve control means. The outflow system is preferably used in conjunction with a fluid delivery system, as described below, to facilitate changing the solution in the tank. The outflow system is connected to the tank at any location that provides suitable drainage, and preferably is located on the bottom half, more preferably on the bottom third, and most preferably on the bottom surface of the microwave tank to facilitate draining. Any suitable outlet can be employed to connect the tank to the outflow system, for example a drain, valve, port, nozzle, adaptor or connector, and can be integrally formed with the tank or can be attached to the tank. In one embodiment, the outlet is a teflon fitting that is attached to an opening on the bottom of the tank and is suitable for connecting to a tube. Alternatively, a suction device can be lowered into the tank to remove the solution, or the tank can be filled from the bottom and an overflow valve can be incorporated in the upper region of the tank. One or more drains can also be incorporated under the tank(s) to further control any overflow, and can feed into the outflow system.

The outflow system can comprise a system as simple as a tray underneath the microwave tank which can receive the fluid released from an outlet on the microwave tank. Preferably, however, the outflow system is enclosed so as to minimize release of volatile components from the draining solutions. The outflow system therefore preferably comprises any conduit 49 having a hollow interior, for example a hose, pipe, or tube. The outflow system is preferably of a size suitable for rapid draining of the microwave tank, preferably comprising tubing of at least about ⅛″ in internal diameter, more preferably at least about ¼″ in internal diameter, or the equivalent. Where a plurality of microwave tanks are employed, the outflow systems from each microwave tank can be connected together, for example via a manifold 51, to minimize the number of downstream components of the outflow system.

In a preferred embodiment, the outflow system comprises silicone or Viton tubing or the equivalent, having a temperature rating of at least about 135° C. The tube then connects to a bulkhead fitting that permits the outflow system to pass through a wall of the microwave chamber. Another tube is then connected to an outer portion of the bulkhead fitting and directs the drainage of the microwave tank solution out of the microwave.

The outflow system preferably incorporates one or more high temperature drainage pumps 53 for more rapid draining of the solution from the tank as shown in FIG. 1c. The outflow valves 47 can be any electronically controllable mechanism for opening and closing an outflow system in the outflow system, and can be located anywhere in the outflow system. The outflow valve control means can be any system which can electronically control the opening and closing of the outflow valve. In a preferred embodiment, the outflow valve control means is a programmable computer system which provides an electronic signal to open and close the outflow valve in conjunction with a computer-controlled drainage pump, for example a valve Snap-Tite Part No. 2W130-INR-V8C6 and pump KNF Newberger Part # PM 13517-ND100TT. The outflow system preferably leads to a waste reservoir for storing the used solution.

Specimen Positioning Device

The apparatus can have one or more specimen positioning devices 55 that can perform any set or subset of the methods described herein as shown in FIG. 3. Although the claims may make reference to either one or multiple specimen positioning devices, it is to be understood that multiple specimen positioning devices can take the place of a single device and a single device can take the place of multiple devices and remain within the scope of the claims unless explicitly stated otherwise, e.g. unless a “single” specimen positioning device or “first and second” specimen positioning devices are explicitly recited in the claims.

The specimen positioning device comprises a positioning element 57, a holding element 59 and holding element control means. The positioning element comprises motors or other means for moving the specimen positioning device under the control of a computer or other electronic control device that allows programming of movement of the specimen positioning device between various work locations on or within the framework. In one embodiment, power is supplied from a step-down autoswitch power supply that can supply either alternating or direct current of different voltages to various components of the apparatus; the distribution of the various currents can be controlled by the multifunction board in the programmable computer system. The positioning element also comprises the associated rails, tracks, or cables along which the motors move. In a preferred embodiment, the positioning element is a three-axis step-motor-driven assembly mounted on rails or tracks. Alternatively, the positioning element can be a single robotics arm.

Visible in FIG. 3 (at the right of the figure) is the X-axis track 61, the X-axis being the principal longer horizontal axis of the apparatus in this embodiment. The single X-axis track is supported at either end on bearing shafts and brackets. The Y-axis 63 is the principal shorter horizontal axis of the embodiment as shown. The Z-axis 65 in this embodiment is the orthogonal vertical axis perpendicular to the plane of the X- and Y-axes. In a preferred embodiment, suitable X-axis and Z-axis tracks are obtained commercially (X-axis Part No. RSR15ZMUU+700LM from THK). The Y-axis shaft system is a linear bearing rail assembly (Part No. RR-PAC0136A manufactured to specification by Pacific Bearing).

The Z-head 67 (FIGS. 3 and 12) component of this embodiment of the specimen positioning device provides the holding element and movement in the Z-direction. Any holding element 59 suitable for moving a specimen between different components of the apparatus can be used, for example a hook, a grasping device, a suction device, or a scoop. Preferably the holding element is a grasping device which can grasp the sides of a rack of specimen slides and maintain a stable, controllable orientation as the rack is moved. In a preferred embodiment, the holding element comprises solenoid-operated grip plates 69 wherein the solenoid 75 directs grasping and releasing motions by the grip plates. In this embodiment, all parts of the Z-head (FIGS. 12 and 13) are manufactured, except for the stepping motors and solenoid. Suitable solenoids are commercially available (McMaster Carr Part Nos. 70155K59 and 69905K28). A sensor can be incorporated to detect the presence of a specimen rack. One embodiment of this sensor is shown in FIG. 12. When the rack 110 is picked up by the specimen positioning device, a part of the rack pushes up the flag 171. The flag 171 disrupts the optical signal in the sensor 174 indicating the presence of a specimen rack 110 held by the gripper 59. If at the end of the pickup sequence the sensor 174 does not sense the flag 171, the system may try to pick up the specimen rack 110 a number of times before giving a warning and calling for user intervention.

Suitable stepping motors 73 for the X-, Y- and Z-axes can be purchased from Oriental Motors (Parts Nos. PK545-NAA, PK566-NBA, PK544-NAA, respectively). The X- and Z-axes motors are modified by adding a cable connector (Molex Part No. 22-01-3057; Molex also supplies a suitable housing (Part. No. 08-50-0114) and crimp terminal (Part No. 08-50-0114) and installing a spur gear (Stockdrive Part No. A1M2MY210012) or pulley (Brecoflex Part No. LS21T5/10-2) on the output shaft.

Specimen Positioning Device and Holding Element Control Means

The specimen positioning device and the holding element are typically operated under the control of a computer or other electronic control device. In the simplest applications, where a single method will be performed repeatedly, it is possible to provide either a hard-wired controller or a non-programmable electronic controller, such as a computer operating under instructions from read-only memory. In preferred embodiments, however, a programmable controller or computer is used so that the operation of the apparatus can be varied.

Preferably a motion controller 79 as known in the art is used in conjunction with a programmable computer as control means for the specimen positioning device and holding element, and must be able to report its position while in motion. In one embodiment, a Galil Motion Control Part No. DC-1738 with optional extra I/O configuration controls the operation of a three-axis step-motor-driven positioning element and the holding element. In conjunction with the specimen identification device 71, the motion controller can assign positions to the specimen without relying on barcode locators.

Fluid Delivery System

As shown in FIG. 1, the fluid delivery system comprises a conduit 81 having a first end for attachment to an outlet on a fluid source and a second end directing output of the conduit into a tank attached to the framework within the apparatus, and fluid delivery control means regulating output from the conduit. The second end of the conduit directs the fluid into the microwave tank in the most basic embodiment, either directly or through intermediate connectors, fittings, conduits, etc. In a preferred embodiment, a plurality of fluid sources each provide a fluid via a plurality of conduits to one or more of the tanks within the apparatus.

The fluid source 83 can be any reservoir or plumbing system that supplies a fluid useful in any of the methods of the invention, and can vary within a single apparatus depending on the tank to be supplied. For example, the fluid source for the rinse tank can be a carboy or other container filled with distilled or deionized water, or can be a distilled or deionized water faucet as can be typically found in a laboratory setting. Where the fluid source is a container, the fluid may be delivered by gravity where the container is elevated relative to the tank being supplied, or the fluid can be delivered by a pump, but the fluid is preferably delivered by a pressure differential such as can be supplied by a compressor 85 connected to an upper portion of the container above the fluid level. The outlet of such a container is preferably located at or near the bottom of the container to facilitate complete drainage of solution from the container. In one embodiment the fluid source comprises a carboy which is pressurized to about five p.s.i. by a computer-controlled compressor, for example model number AC0105 from Medo Company. The compressor pressure line can be connected through a manifold or similar device to a plurality of containers as shown in FIG. 1. A pressure gauge 87 and pressure relief valve 89 or a switch can be included so as to prevent excess pressure from building up in the system, which could lead to undesired fluid leaks and fluid delivery failure.

A plurality of fluid sources are employed in a preferred embodiment of the invention to provide different solutions for the microwave tank(s), the holding tank and the rinsing tank. A plurality of different fluid sources can also be employed to allow rapid switching between different fluid sources that supply a given tank. For example, a plurality of different one-step combined antigen retrieval and dewax solutions can be provided to allow for selection of a preferred solution in conjunction with immunostaining using a particular antibody. Conversely, a single fluid source can be supplied to a plurality of tanks via a manifold 91; for example, one antigen retrieval fluid source can have an outlet connected to a conduit comprising a manifold having a plurality of second ends directing the distribution of antigen retrieval fluid to a plurality of microwave tanks, each second end having a fluid delivery control means.

The conduit can comprise any tube, pipe or hose (as described above with reference to the microwave tank outflow system) that leads from the fluid source to or towards a tank within the apparatus. The conduit may comprise intermediate connectors, for example bulkhead fittings passing through the walls of the microwave chamber. Preferably the conduit in the fluid delivery system is also Viton or silicone tubing or the equivalent, as described above. The first end of the conduit is attached to the fluid source in any acceptable manner, for example via a fitting. The fluid delivery control means can be any automated mechanism for opening and closing the conduit leading from the fluid source to a tank within the apparatus. In a preferred embodiment, the fluid delivery control means comprises an electrically operated valve 93 whose opening and closing is controlled by a programmable computer via a multifunction board 79 (FIGS. 1 and 3).

Another feature of the system is the fluid level detection in any of the tanks described here and below, i.e. microwave tanks 5, rinse tank 15, and holding tanks 19. This detection system is shown in FIG. 12. In general operation the present apparatus can detect the fluid level in each of the above mentioned tanks. The components of the system are: a pressure sensor 181, a controlled static air pressure supply 183, and a computer based data acquisition system 187. A steady low pressure (0.5 to 1 psi) of compressed air is supplied from the air supply 183 through a tip 189. The tip is held stationary at a predetermined level of the tanks. At the same time the computer is continuously sampling the pressure data from the sensor 181 with the data samples being averaged to eliminate extraneous noise and sample error. At this time the tank being monitored is being filled with fluid. When the liquid level reaches the tip 189 a back pressure is created and the pressure change is sensed by the pressure sensor 181. If the level is not sensed in a given period of time the computer interprets this as an error and flashes a message for user intervention.

Alternately, level of the fluid can be detected in the reagent vials using an optical or an electrical probe integrated with reagent tip head 189. In the optical methodology fluid level may be attained by disrupting a light beam which is detected by the optical sensor. In the electrical methodology fluid level may be ascertained by a current flow due to the presence of the reagent. Further, a sensor 188 can be provided in the fluid reservoir to monitor the fluid level. The sensor is positioned at the bottom of the reservoir. A similar sensor 186 can be provided in the waste carboy, positioned at the top of the carboy to sense when the carboy is full and needs to be replaced.

Rinse Tank and Holding Tank

The apparatus of the invention also preferably comprises other tanks suitable for permitting other steps to be performed on specimens. One or more rinse tanks 15 and one or more holding tanks 19 are preferably included in the apparatus, as shown in FIGS. 1 and 10, and can be formed individually or provided as an integrated unit 95, as shown in FIG. 11. A rinse tank can be provided for rinsing specimens, and can be manufactured in a similar manner to the microwave tank as described above, although it need not be manufactured of microwave-safe material, as it is generally located outside of the microwave chamber attached to the framework of the apparatus, either directly or indirectly, and is accessible to the specimen positioning device. The rinse tank can be shaped so as to accommodate a variety of different slide rack shapes and sizes. The tank can be attached via engagement within a receptacle for positioning it within the apparatus. In one embodiment, the rinse tank is manufactured of stainless steel. As with the microwave tank, the rinse tank can also comprise an outflow system and be supplied by a fluid delivery system, which can be separate from or integrated with the fluid delivery system supply and outflow for the microwave tank(s).

The rinsing solution is typically distilled or de-ionized water, although any suitable solution can be used, containing for example a buffer, salts, detergents, surfactants, etc. One embodiment of a rinse tank is shown in FIG. 9, showing a rinse tank suitable for accommodating one slide rack and having fittings 97 for attaching tubes from a fluid delivery system and an outflow system for filling and draining the tank with rinsing solution. A suitable rinse tank can be manufactured to specification by an approved vendor.

A holding tank can be provided for retaining specimens after dewaxing or antigen retrieval. The holding tank preferably has a plurality of compartments for holding different specimens. The compartments can be separated by dividers that allow less holding solution to be used when less than all the compartments are to be occupied by processed specimens. Alternatively, one or a plurality of holding tanks each suitable for accommodating a single slide rack can be provided, and can be shaped so as to accommodate a variety of different slide rack shapes and sizes. The holding tank can be manufactured in a similar manner to the microwave tank as described above, although it need not be manufactured of microwave-safe material, as it is generally located outside of the microwave chamber attached to the framework of the apparatus, either directly or indirectly, and is accessible to the specimen positioning device. The tank can be attached via engagement within a receptacle for positioning it within the apparatus. In one embodiment, the holding tank is manufactured of stainless steel. As with the microwave tank and the rinse tank, the holding tank, or the separated compartments, can comprise an outflow system and a fluid delivery system, which can be separate from or integrated with those of the microwave tank(s). The holding tank can be integrally formed with the rinse tank and separated by a divider, or the holding tank can be separate from the rinse tank. One embodiment of the holding tank is shown in FIG. 10, demonstrating a tank suitable for holding a plurality of slide racks and having fittings 99 for attaching tubes from the fluid delivery system and outflow system. A suitable holding tank can be manufactured from stainless steel to specification by an approved vendor.

The holding solution can be any solution suitable for storing the specimens after dewaxing or antigen retrieval, but is preferably a buffered solution, for example phosphate-buffered saline (PBS), that allows for the subsequent performance of antibody staining techniques. Buffers generally provide a pH of 6.5 to 8.5, preferably about 6.8 to 8.0, and most preferably about 7.0 to 7.6. Numerous physiological buffers are commercially available through biological supply houses. Specific buffers may be selected according to the antibody being used.

Staining Tanks

The apparatus can also include a tank or tanks for performing various staining methods, and, where several tanks are used, they may be individually or integrally formed. Preferably, the apparatus comprises a hematoxylin tank, an eosin tank and a differentiation tank for performing hematoxylin and eosin staining. The staining tanks can be formed in a similar manner to the microwave tanks, but need not be made of microwave-safe material, as they are typically located within the framework of the apparatus outside of the microwave chamber, and are accessible to the specimen positioning device. These tanks can be fixed or removable, and can be attached directly or indirectly to the framework. The tank can be attached via engagement within a receptacle for positioning it within the apparatus. The staining tanks may or may not comprise a fluid delivery system or an outflow system, as the staining solutions generally do not require frequent changing.

One embodiment of a staining tank 17 is shown in FIG. 8. The tank is generally rectangular in cross-section so as to accommodate typical rectangular slide racks, and can be shaped so as to accommodate a variety of different slide rack shapes and sizes. Dehydration and cleaning of slides can be accomplished inside the system by provision of additional tanks. These additional tanks may contain inflammable or volatile organic chemicals which require automated or mechanically removable lids to reduce evaporation and potential hazards.

Loading Area

The apparatus preferably includes a loading area 101 for storage of a plurality of specimens prior to automated dewaxing, antigen retrieval or staining as shown in FIG. 4. The loading area is located outside of the microwave chamber and is preferably attached to or integrally formed from the supporting framework. The loading area may also be used for storage of specimens after automated procedures have been performed. Different types of slide racks can be accommodated by suitable modifications to the loading area and microwave tanks. The loading area can also be used for air drying specimens and for storing specimens after a staining method, for example H&E staining. The loading area can comprise a drain, which may drain into a common waste reservoir, to drain any fluid released from the specimen during storing or air drying. In one embodiment, the loading area is used for storing specimens after H&E staining to minimize the footprint of the device. The loading area as shown in FIG. 18 can store a large number of slide racks pre- and post-processing. Thus, a large number of slides can be processed an stored on one run. This results in very effective high throughput, walk away automation system. The design also lends itself to continuous feed processing.

Specimen Identification Device

The apparatus also preferably comprises a specimen identification device 71 that can track the location and identity of individual specimens within the apparatus as shown in FIG. 3. Additionally, the specimen identification device can prevent the subjection of specimens to undesired methods. The specimen identification device is preferably attached to the specimen positioning device so that specimen positioning and detection can be integrated. The specimen identification device can be any device which can detect the specimen by detecting, directly or indirectly, the specimen itself or a material connected to the specimen. Detection can be accomplished, for example, optically, magnetically or electrically. In a preferred embodiment, the specimen identification device comprises an optical scanning device, for example a barcode scanner (Keyence Part. No. BL600-H) which is operated by a programmable computer system, and an operating program. The scanner may be suitably shielded so that only one specimen is read at a time to allow identification of unique specimens and assignment of unique positions.

Where the specimen is mounted on a support, such as a slide, comprising a barcode, the barcode scanner can then detect the presence of the specimen, determine which pre-programmed processing protocol is designated by the barcode, and identify its location in combination with the system software and computer-controlled specimen positioning device. If a specimen is found to be in an undesired location, for example in an improper tank at the start of an operating sequence or in a rack with specimens requiring a different processing protocol, the system can identify the offending specimen so that the user can remove it. Alternatively, the system can be programmed to remove a detected rack from an undesirable location and move it to a preferable location, for example where it can be removed by the user or kept out of the way while other racks are processed.

In conjunction with the optical scanning device, a microscope slide having an optically detectable identification label on an edge is also provided. This allows the slide to be detected in a vertical position, and so allows for detection of the slide in a higher density setting than as typically labeled. Preferably, the identification label is attached to one of the shorter edges of the slide so that the slide can be detected from above in a standard slide rack. Where the detection device is an optical scanning device, the identification label is optically detectable. Preferably the identification label is a barcode. The barcode can be a combined barcode which can also be read by the BioGenex automated staining system and the two devices can also share information related to the specimen, including specimen identification and protocol identification. The barcode can be affixed to the slide via a pre-printed label or can be manufactured on the slide.

General Apparatus Considerations

The apparatus of the invention can generally be prepared from readily available commercial parts and requires few specially manufactured parts. Microwave units, rails, motors, electrical connections, tubing, compressors, liquid distribution systems, and many other components are commercially available from a variety of suppliers. The composition of the components from which various parts are manufactured can vary widely, but components which contact potentially corrosive reagent or wash solutions are typically prepared from or coated with resistant materials, for example stainless steel, glass, ceramic, teflon or inert plastic. For example, the material used for fluid delivery and outflow system tubing can be selected individually for specific liquid solutions to which they will be exposed; the tubing used for dewaxing solutions should be resistant to organic solvents and detergents (Viton-type or silicone tubing).

The various control means used in the apparatus for the specimen positioning device, the holding element, the fluid delivery system, and the outflow valve(s) as well as the microwave chamber sealing means can be integrated on a single programmable computer 103 and can be operated by one or more circuits in conjunction with one or more programs on the computer as shown in FIG. 3. Although separate computers or electronic controllers can be used for each control or sealing means and are within the scope of the claims, integration on a single computer is preferred in order to simplify the apparatus. Such a system preferably employs a computer having at a minimum a Pentium® processor or equivalent, and advantageously can run the Windows NT® operating system. A multifunction board 35 can be designed to provide the various control and sealing means in conjunction with a computer 103 and an I/O interface board 105 (FIG. 3).

Software will generally be provided with the computer so that the user does not need to provide instructions for individual motions, but merely selects appropriate protocols from a menu. However, the apparatus can operate in an ‘open’ format in which the user is asked to supply various parameters, for example the length of time for various steps; all other operations are carried out by pre-programmed instructions in the computer, which directs movement of the specimen positioning device to the appropriate locations and controls the operation of the microwave and other components.

In a ‘closed’ format, barcode or equivalent technology can be used to supply instructions to the apparatus. The apparatus reads barcodes associated with the specimens; thereafter, the computer is able to determine all parameters needed to carry out the most appropriate pre-programmed instruction set in the memory of the computer to control the apparatus in the processing procedures for microscope slide staining. Compared with the ‘open’ format, less user input is required, thus reducing the opportunities for introduction of error. It is especially useful for those users who perform large batch procedures. This mode of system operation is simple and can be performed by a laboratory technician. The apparatus of the invention can contain additional components and subsystems for convenience. For example, drain trays with exit conduits to waste reservoirs can be located either individually under components of the apparatus or a single drain tray and collection system can be provided for the entire interior space of the apparatus frame in order to control drips or spills.

General Operation of the Apparatus

The apparatus is designed so that, once all components are in place, the apparatus can automatically carry out all positioning, heating, incubation, and rinsing steps to perform the desired method. In a preferred embodiment, before initiating an operation, the user loads one or more specimens, typically attached to slides placed in a rack, in the loading area 101 (FIGS. 4 and 5) of the apparatus. The user then initiates an operating sequence. The apparatus moves a specimen identification device 71 (FIGS. 3 and 12) around the apparatus to detect specimens within the apparatus by their identification labels and then determines if all the specimens within a given rack require the same treatment. If all the specimens in a rack do not require the same treatment, the apparatus will prompt the user so that the nonconforming specimen(s) can be removed. At the same time, if any unknown identification label is detected, the computer will request that the user create a new protocol. Additionally, if specimens are identified within the apparatus at the start of an operation cycle at positions other than the loading area, the apparatus will prompt the user to remove them. After this verification, the computer will control the apparatus to automatically process the specimen.

In a typical operating sequence, the specimen positioning device is moved to different locations within the apparatus by the action of various motors that operate in combination with sliding tracks to precisely position the specimen positioning device at its desired location within the framework, in order to carry out histotechnological methods on the specimen. The positioning device moves the specimen between the loading area 101, the microwave tank(s) 5, the rinse tank 15, the staining tank(s) 17 and the holding tank 19, as appropriate for the designated method, and can perform additional operations programmed by the user.

In a dewaxing, antigen retrieval, or combined dewaxing and antigen retrieval method, the specimen positioning device places the specimen in a microwave tank 5. The specimen is contacted with a solution appropriate for the designated method in the microwave tank. The microwave chamber is then sealed, and microwaves are directed into the microwave chamber to heat the solution. The heating time is determined in accordance with the method chosen. A series of heating cycles can be performed with one or more intervening changes of solution in the microwave tank where desirable.

After the heating sequence is complete, the chamber is unsealed. For methods including an antigen retrieval step, the specimen is allowed to remain in the solution while it cools, during which time further antigen retrieval occurs. The specimen is retrieved from the microwave tank by the specimen positioning device 55 and placed in the rinse tank 15. The specimen is then contacted with a rinse solution in the rinse tank. A rinsing sequence takes place in which the specimen can be dipped in the rinse solution repeatedly by the specimen positioning device, and one or more changes of rinse solution can occur. The specimen can then be placed in a storage area until the operation cycle is completed for all the specimens. Preferably the storage area is a holding tank 19, which preferably contains a buffered solution suitable for storing the specimen prior to performing further histotechnological methods.

For a staining operation, the specimen positioning device moves the specimen from the loading area or from dewaxing tanks to a staining tank 17. Depending on the precise staining technique being used, a plurality of staining and/or destaining tanks can be employed. Destaining steps, for regressive staining methods, as well as rinse steps can also be incorporated in the staining operation. After staining, the specimen can then be stored as appropriate for the stain being used, for example the loading area 101 or a holding tank 19 without holding solution. For most stains, the specimen should not be stored in solution so that the stain is not lost from the specimen.

Automated Dewax Method

The automated method of specimen dewaxing of the invention comprises transporting the specimen into a tank within a microwave using a computer-controlled specimen positioning device, providing a suitable dewaxing solution in the tank, and heating the solution using a computer-controlled microwave unit. The process of heating in the solution can be repeated for proper deparaffinization of the specimen. A temperature above the melting point of paraffin (56° C.) is typically used for dewaxing, and preferably is much higher. The solution is preferably heated so that it maintains a temperature of at least about 60° C. and is kept below the solution boiling point for at least about 3 minutes. The period of time during which this elevated temperature is maintained is referred to as “simmering.” Preferably the solution maintains a temperature of about 99° C. or less during simmering. Automated dewaxing can be performed alone or in a combined method in conjunction with automated antigen retrieval or specimen staining.

The dewaxing solution can, for example, be one described in U.S. patent application Ser. No. 08/212,175 to Zhang et al. entitled “Deparaffinization Compositions and Methods for their Use” and filed Mar. 11, 1994. In particular embodiments, EZ-DeWax™ Ready-To-Use or diluted EZ-DeWax™ Concentrate, both available from BioGenex Laboratories, can be used. In a preferred embodiment, the dewaxing solution comprises EZ-DeWax™ Ready-To-Use diluted in water. The diluted dewaxing solution preferably comprises: from about 0.3 to about 15% isoparaffin, more preferably about 3%; from about 0.3 to about 15% of a C1 to C5 alcohol, more preferably about 3%; from about 0.01 to about 5% TritonX-100, more preferably about 0.1%; from about 0.006 to about 1% Brij35, more preferably about 0.06%; from about 0.0003 to about 1% SDS, more preferably about 0.03%; and 0.005 to about 0.5 M sodium citrate, more preferably about 0.05 M. Where simultaneous dewaxing and antigen retrieval are desired, the solution also comprises compounds which permit antigen retrieval as described below.

The concentration of the dewaxing solution and the temperature at which dewaxing occurs are inversely related. Dewaxing can be performed at any temperature from room temperature to about 98° C. by adjusting the concentration; higher temperature require lower concentrations of dewaxing solution to provide satisfactory dewaxing.

The method preferably also comprises a step in which the solution is replaced with fresh dewaxing or dewaxing and antigen retrieval solution, which can be the same or different as the solution used in the first heating step, and the heating process repeated to obtain optimum removal of embedding material from the specimen. (As used herein, the term “fresh” refers to a new batch of solution and does not necessarily imply that the solution is freshly made.) The second simmering period can vary depending on whether the specimen is to undergo only dewaxing, or both dewaxing and antigen retrieval by using a combined antigen retrieval and dewax solution. If only dewaxing is to occur, the second simmering period is preferably about 3 minutes, and typically from about 3 to about 10 minutes. If antigen retrieval is also to occur, the second simmering period is preferably at least about 15 minutes, and more preferably about 20 minutes, but preferably does not extend so long that the immunoreactivity of the specimen decreases, or that the specimens are lost from the slides, or tissue integrity is adversely affected.

After dewaxing, the specimen is preferably rinsed. The specimen positioning device retrieves the specimen from the microwave tank, which is preferably first drained to assist in removal of the dewaxing solution, and transports it to a rinse tank. The specimen is preferably first held above the microwave tank by the positioning device for a short period of time to limit the dripping of solution onto other portions of the apparatus during transit. The exact period of time the specimen is held above the tank is not critical, but is typically at least about five seconds, and more typically at least about ten seconds. This period of time preferably does not extend for so long that the method is greatly lengthened or the specimen is allowed to become dry, ceasing when most of the dripping has stopped, which is typically about one minute or less, and preferably is about 30 seconds or less.

The rinse solution can be any solution that does not adversely affect the specimen or the remaining steps to be performed on the specimen, and is typically distilled or deionized water, but can include components including buffers, detergents, surfactants, and/or chelating agents where desired.

Rinsing preferably includes at least one change of solution within the rinse tank, and can include a continuous exchange while the specimen is in the tank. Two rinses with one change of solution in between are typically sufficient, however. Rinsing is preferably aided by raising and lowering (“dipping”) the specimen in the rinse tank using the specimen positioning device; the exact number of dips is not critical, but preferably is at least three, and more preferably is about five. Preferably the number of dips is not so great that it adversely affects the specimen or unduly increases the time of the method.

After rinsing, the specimen is preferably placed in a holding tank, and the specimen positioning device proceeds to the next specimen, if any. The holding tank is preferably filled with a solution that does not adversely affect the dewaxed specimen, and is preferably a buffered solution compatible with immunochemical methods or in situ hybridizations to be performed on the specimen, for example phosphate-buffered saline.

Automated Antigen Retrieval Method

The automated method of antigen retrieval of the invention comprises transporting the specimen into a tank within a microwave using a computer-controlled specimen positioning device, providing an antigen retrieval solution in the tank, and heating the solution using a computer-controlled microwave unit. The solution is preferably heated so that it maintains a temperature of from about 50° C. to at or just below the solution boiling point, 99° C., for a time sufficient to enhance immunostaining of the specimen, typically for at least about 15 minutes, and preferably at least about 20 to 30 minutes, but preferably not so long as to adversely affect the specimen or unduly increase the length of the method. Lower temperatures can also be used, but require additional time for antigen retrieval, and may not provide satisfactory antigen retrieval if the temperature is too low. Different antigens or antibodies may require different simmering times, and these requirements can also vary depending on the nature of the antigen retrieval solution. Appropriate simmering times are known to or can be determined empirically by one of skill in the art. Preferably the solution maintains a temperature of about 99° C. or less during simmering. After heating, the specimen is allowed to cool in the solution in the microwave tank, preferably for about 20 to about 30 minutes at ambient temperature. The microwave chamber is preferably unsealed during cooling. The temperature to which the materials cool is not particularly important. However, the gradual cooling process is critical for optimum antigen retrieval.

Where a combined dewaxing and antigen retrieval method is to be performed, the method includes a step in which the solution is replaced and the heating process repeated to obtain optimum removal of embedding material from the specimen. The first simmering period in such a case is preferably at least about 3 minutes to about 10 minutes, and the second simmering period is as described above, but preferably the total simmering time does not extend so long that the immunoreactivity of the specimen decreases, or that the specimens are lost from the slides, or that tissue integrity is adversely affected.

It is preferable to carry out the microwave heating step in an aqueous solution comprising at least one component which results in increased recovery of antigens in a specimen when heated with microwave radiation. Preferably, the antigen retrieval solution comprises chelating agents, for example EDTA, EGTA, or citrate salts, or metal ions, derived for example from salts of lead or zinc ions as described in U.S. Pat. No. 5,578,452 to Key et al. and issued Nov. 26, 1996. Other useful antigen retrieval solutions include Citra (Cat. No. HK087-5K), Citra Plus (Cat. No. HK081-5K), AR-10 (Cat. No. HK058-5K) and Glyca (Cat. No. HK166-5K), all available from BioGenex Laboratories, Inc., San Ramon, Calif. Any concentration of chelating agents or metal ions can be used which results in increased recovery of antigens in the specimen as compared to heating in water alone. Where EDTA is used, it is typically present at about 0.01 to about 10 mm, more preferably about 0.05 to about 2 mm, and in one embodiment at about 0.1 mm. Where citrate solution is used, it is typically present at a concentration of about 0.001 to about 0.5 M, preferably at about 0.05 M, and has a pH in the range of about 4 to about 10, preferably about 6. A Tris-based solution can be used, having a concentration of from about 0.001 to about 0.5 M Tris, preferably about 0.01 M, and a pH of from about 8 to about 12, and preferably about 10.5. The pH of the antigen retrieval solution can be adjusted to optimize antigen recovery for the particular antigen, specimen and antibody, and determining a suitable pH is within the skill of the art.

For combined dewaxing and antigen retrieval, the solution also comprises dewaxing components as described above.

After antigen retrieval, or combined dewaxing and antigen retrieval, the specimen is preferably rinsed and then transferred to the holding tank as described above.

Automated Hematoxylin and Eosin Staining Method

The automated method of hematoxylin and eosin staining of the invention comprises transporting the specimen into a hematoxylin tank using a computer-controlled specimen positioning device, providing a hematoxylin solution in the tank, and contacting the specimen with solution for a time sufficient to stain it with hematoxylin, typically by immersion. The specimen is also transported into an eosin tank using the computer-controlled specimen positioning device, providing an eosin solution in the tank, and contacting the specimen in the solution for a time sufficient to stain it with eosin. The staining steps can be performed in an order where typically hematoxylin staining is performed before eosin staining. Preferably, the staining method is a regressive one in which the specimen is overstained and then treated with a differentiation solution which decolorizes regions of the specimen whose staining is not desired, and then treated with a clearing solution to prepare the specimen for permanent mounting. H&E staining can be performed alone or in combination with other methods of the invention.

Where a combined dewaxing and H&E method is to be performed, an initial dewaxing step is followed by a step in which the solution is replaced and the heating process repeated to obtain optimum removal of embedding material from the specimen, as described above. After rinsing, the H&E method can then be performed.

Hematoxylin, eosin and differentiation solutions are known, and appropriate solutions for a particular embodiment can be selected by one of skill in the art. In one embodiment, the hematoxylin solution comprises hematoxylin, ammonium alum, NaIO₃, glycerol and glacial acetic acid. The hematoxylin is typically present at about 0.05 to about 10%, more preferably about 0.1 to about 1%. Ammonium alum is typically present at about 0.05 to about 25%, preferably about 1.0 to about 10%. NaIO₃ is typically present at about 0.001 to about 5%, preferably about 0.01 to about 1%. Glycerol is typically present at about 5 to about 60%, preferably from about 10 to about 50%. Glacial acetic acid is typically present at about 0.1 to about 10%, preferably from about 0.5 to about 5%.

In one embodiment, the eosin solution comprises Eosin Y and reagent alcohol in distilled water. The Eosin Y is typically present at about 0.05 to about 10%, preferably from about 0.5 to about 2%. The reagent alcohol is typically present at about 30 to about 90%, preferably from about 60 to about 80%.

The differentiation solution typically comprises from about 0.01 to about 5% ammonium hydroxide, preferably about 1%.

Preferably there is a rinsing step between the staining steps, and between the staining and differentiation.

After H&E staining, or combined dewaxing and H&E staining, the specimen is preferably rinsed and then transferred to a dry storage area, as the stains could be washed from the specimen if it were stored in solution. In one embodiment, the H&E stained specimen is transferred to an open position in the loading area (FIG. 1).

Rinses

It is desirable to rinse the specimen between contacting different solutions or upon completion of the automated dewaxing, antigen retrieval or staining methods described herein. Although it is possible that the specimen could be transferred from one solution directly to the next, rinsing is preferred in order to minimize specimen background and maximize the useful lifetime of the solutions. The specimen is preferably rinsed a plurality of times, which can include a change of rinsing solution, multiple submersions and retrievals (“dips”) of the specimen(s) in the rinsing solution, or both. The rinses are typically performed with distilled or deionized water, although any suitable solution can be used, containing for example a buffer, salts, detergents, surfactants, etc.

Further Processing

The specimen can be subjected to further processing steps prior to analysis. The specimen can be dehydrated, for example by treatment with 100% alcohol or a mixture of alcohols, or a graded series of alcohols containing decreasing amounts of water. Preferably, the alcohols are lower alcohols containing from one to six carbons, more preferably from one to four carbons. The specimen can be cleared, for example by exposure to xylene. Additional staining methods can also be performed. In some instances, it can be desirable to later treat the specimen so as to remove the effects of prior processing steps and then perform different processing steps incompatible with the steps initially performed. For example, it may be desirable to remove a stain from a given specimen and perform a different staining procedure on the specimen to visualize a different specimen component. Finally, the specimen can be mounted with any suitable mounting media, for example Permount.

Automated Methods of Additional Processing

The device of the invention can be linked to another device that can perform additional histotechnological methods on the specimen, for example immunohistochemistry, in situ hybridization, or other staining methods. Any device which can perform such methods on the specimen can be used, for example the OptiMax® or the OptiMax Plus® (U.S. Pat. Nos. 5,439,649 and 5,948,359), or the GenoMax™ 6000 and i6000™ (patent pending).

### EXAMPLES

The following examples are set forth so as to provide those of ordinary skill in the art with a complete description of how to make and use the present invention, and are not intended to limit the scope of what is regarded as the invention. Unless indicated otherwise, parts are parts by weight, temperature is degree centigrade and pressure is at or near atmospheric, and all materials whose catalog numbers are indicated are available from BioGenex Laboratories, Inc., San Ramon, Calif.

### Example 1

### Preparation of One-Step Antigen Retrieval and Dewaxing Solution 1

One-Step Antigen Retrieval and Dewaxing Solution 1 (“AR1”), a preferred solution, was prepared according to the following protocol:

| | | |
|---|---|---|
| EZ-Dewax (HK585-5K) | 37.5 | ml |
| Antigen Retrieval Citra Plus (HK080-9K) 10X concentrate | 50 | ml |
| (pH 6.0) | | |
| Distilled H₂O | 412.5 | ml |
| Total volume | 500 | ml. |

The solution was stored at room temperature.

### Example 2

### Preparation of One-Step Antigen Retrieval and Dewaxing Solution 2

One-Step Antigen Retrieval and Dewaxing Solution 2 (“AR2”), a preferred solution, was prepared according to the following protocol:

| | | |
|---|---|---|
| EZ-Dewax (HK585-5K) | 37.5 | ml |
| Antigen Retrieval Citra Plus (HK080-9K) 10X concentrate | 50 | ml |
| (Adjust pH to 8.4 using 5M NaOH) | | |
| Distilled H₂O | 412.5 | ml |
| Total volume | 500 | ml. |

The Citra Plus concentrate was added to 200 mls of the distilled water, and the pH was adjusted to 8.4 using 5M NaOH. Then the EZ-Dewax solution was added, and the remainder of the distilled water was added. The solution was stored at room temperature.

### Example 3

### Preparation of One-Step Antigen Retrieval and Dewaxing Solution 3

One-Step Antigen Retrieval and Dewaxing Solution 3 (“AR3”), a preferred solution, was prepared according to the following protocol:

| | | |
|---|---|---|
| For 1 mm EDTA (pH 8.0), add 0.37 gms. of EDTA | 50 | ml |
| to 100 ml. of distilled water. | | |
| (Adjust pH to 8.0 using 5.0 M NaOH) | | |
| EZ-Dewax (HK585-5K) | 37.5 | ml |
| Distilled H₂O | 412.5 | ml |
| Total volume | 500 | ml. |

### Example 4

### Preparation of One-Step Antigen Retrieval and Dewaxing Solution 4

One-Step Antigen Retrieval and Dewaxing Solution 4 was prepared according to the following protocol:

| | | |
|---|---|---|
| EZ-Dewax (HK585-SK) | 37.5 | ml |
| Antigen Retrieval Glyca Soln. (HK167-5K), 10X concentrate | 50 | ml |
| (Adjust pH to 3.0 using 1M HCl) | | |
| Distilled H₂O | 412.5 | ml |
| Total volume | 500 | ml. |

### Example 5

### Preparation of One-Step Antigen Retrieval and Dewaxing Solution 5

One-Step Antigen Retrieval and Dewaxing Solution 5 was prepared according to the following protocol:

| | | |
|---|---|---|
| EZ-Dewax (HK585-5K) | 37.5 | ml |
| Antigen Retrieval AR-10 Soln. (HK057-SK), 10X concentrate | 50 | ml |
| (Adjust pH to 10.0 using 5M NaOH) | | |
| Distilled H₂O | 412.5 | ml |
| Total volume | 500 | ml. |

### Example 6

### Automated Method for Dewaxing a Specimen

The following protocol outlines the operation of a preferred embodiment of the device of the invention to automatically dewax a rack of barcoded specimen slides:

1. Read the barcode of all the slides inside the specified rack and prompt the user if all the slides are not intended to undergo the same procedure.

2. Identify the position of the slide in the slide rack having the inappropriate barcode so that it can be removed and the remaining slides processed.

3. Move the slide rack into the microwave tank.

4. Fill the tank with a solution comprising a dewaxing agent.

5. Seal the microwave chamber.

6. Heat the solution to 98° C. and maintain the temperature for 3 minutes (total time of 10-12 minutes).

7. Stop heating.

8. Drain and refill the tank.

9. Repeat steps 6 & 7.

10. Drain the tank.

11. Keep the rack hanging above the tank for 10 seconds to reduce the liquid dripping over other parts of the system.

12. Wash the slides in water in the rinse tank 2 times with 5 up and down motions each time, changing the water between each wash.

13. Place the slide rack in the Holding tank filled with buffer.

### Example 7

### Automated Method for Dewaxing and Staining a Specimen with Hematoxylin and Eosin

The following protocol outlines the operation of a preferred embodiment of the device of the invention to automatically dewax and stain a rack of barcoded specimen slides:

1. Read the barcode of all the slides inside the specified rack and prompt the user if all the slides are not intended to undergo the same procedure.

2. Identify the position of the slide in the slide rack having the inappropriate barcode so that it can be removed and the remaining slides processed.

3. Move the slide rack into the microwave tank.

4. Fill the tank with a solution comprising a dewaxing agent.

5. Seal the microwave chamber.

6. Heat the solution to 98° C. and maintain the temperature for 3 minutes (total time of 10-12 minutes).

7. Stop heating.

8. Drain and refill the tank.

9. Repeat steps 6 & 7.

10. Drain the microwave tank.

11. Keep the rack hanging above the tank for 10 seconds, to reduce the liquid dripping over other parts of the system.

12. Wash the slides in water in the rinse tank 2 times with 5 up and down motions each time, changing the water between each wash.

13. Move the slide rack to the hematoxylin tank and immerse it for 5 minutes.

14. Keep the rack hanging above the tank for 30 seconds, to reduce the liquid dripping over other parts of the system.

15. Wash the slides in water in the rinse tank 3 times with 5 up and down motions each time, changing the water between each wash.

16. Move the slide rack to the eosin tank and immerse it for 3 minutes.

17. Keep the rack hanging above the tank for 30 seconds to reduce the liquid dripping over other parts of the system.

18. Wash the slides in water in the rinse tank 2 times with 5 up and down motions each time, changing the water between each wash.

19. Move the slide rack to the differentiation tank and immerse it for 2 minutes.

20. Keep the rack hanging above the tank for 30 seconds, to reduce the liquid dripping over other parts of the system.

21. Wash the slides in water in the rinse tank 1 time with 5 up and down motions.

22. Move the slide rack to the loading area.

### Example 8

### Automated Method for Simultaneous Specimen Dewaxing and Antigen Retrieval

The following protocol outlines the operation of a preferred embodiment of the device of the invention to automatically dewax and retrieve antigens in a rack of barcoded specimen slides:

1. Read the barcode of all the slides inside the specified rack and prompt the user if all the slides are not intended to undergo the same procedure.

2. Identify the position of the slide in the slide rack having the inappropriate barcode so that it can be removed and the remaining slides processed.

3. Move the slide rack into the microwave tank.

4. Fill the tank with a combined dewax and antigen retrieval solution.

5. Seal the microwave chamber.

6. Heat the solution to 98° C. and maintain the temperature for 3 minutes (total time of˜10-12 minutes).

7. Stop heating.

8. Drain and refill the tank.

9. Heat the solution to 98° C. and maintain the temperature for 20 minutes.

10. Stop heating.

11. Unseal the microwave chamber and allow the solution to cool for 20 minutes. Do not drain the microwave tanks at this time as the slides should cool down while immersed in antigen retrieval solution.

12. Drain the tank.

13. Keep the rack hanging above the tank for 10 seconds, to reduce the liquid dripping over other parts of the system.

14. Wash the slides in water in the rinse tank 2 times with 5 up and down motions each time, changing the water between each wash.

15. Move the rack to the holding tank.

### Example 9

### Determination of Microwave Antigen Retrieval Heating Time

A series of experiments were performed in order to determine the amount of microwave heating required for retrieval of antigens in a specimen. Tables 1, 2 and 3 show the effect of different simmering times on antigen retrieval in formaldehyde-fixed, paraffin-embedded tissue specimens for three antibodies: AM256, AM328, and AM370, all available from BioGenex Laboratories, Inc. Solution AR1 (Example 1) containing the specimens was heated until 98° C. was achieved and then maintained for three minutes. The solution was changed, and the fresh AR1 solution was heated to maintain a temperature of about 98° C. to about 99° C. (“simmering”) for various periods of time. Staining of the specimens was graded on a 1 to 4.5 scale, with 4.5 being the most intense, and N referring to not detectable. At 12 minutes of simmering, there was no detectable improvement in antigen retrieval with the three antibodies. At 15 minutes of simmering, two of the antibodies gave strong staining, with the third giving an intermediate level of staining. At 20 minutes of simmering, all three antibodies gave strong staining. A second simmering time of at least about 20 minutes was identified as the minimum required to maximize antigenicity for the broadest range of antibodies tested using AR1 as the antigen retrieval solution.

TABLE 1
ANTIBODY STAINING RESULTS OBTAINED WITH A SIMMERING TIME OF 12 MINS.

| S | | ANTIBODY | | | PREVIOUS | RESULTS | RESULTS |
|---|---|---|---|---|---|---|---|
| # | ANTI # | NAME | LOT. | TISSUE | PROTOCOL | SLIDE 1 | SLIDE 2 |
| 1 | AM256 | Androgen | 2561095 | Prostate CA | Citra 30 m, RT | N | N |
| | | receptor | | | | | |
| 2 | AM325 | Progesterone | 3280898C | Breast CA | Citra 30 m, RT | N | N |
| | | receptor | | | | | |
| 3 | AM370 | Ki-67 antigen, | 3700398 | Tonsil | Citra 2 h, 370 C. | N | N |
| | | Proliferating cell | | | | | |
| | | (Ki88) | | | | | |

TABLE 2
ANTIBODY STAINING RESULTS OBTAINED WITH A SIMMERING TIME OF 15 MINS.

| S | | ANTIBODY | | | PREVIOUS | RESULTS | RESULTS |
|---|---|---|---|---|---|---|---|
| # | ANTI # | NAME | LOT. | TISSUE | PROTOCOL | SLIDE 1 | SLIDE 2 |
| 1 | AM256 | Androgen | 2561095 | Prostate CA | Citra 30 m, RT | 2 | 2 |
| | | receptor | | | | | |
| 2 | AM328 | Progesterone | 3280898C | Breast CA | Citra 30 m, RT | 4 | 4 |
| | | receptor | | | | | |
| 3 | AM370 | Ki-67 antigen, | 3700398 | Tonsil | Citra 2 h, 370 C. | 4.5 | 4.5 |
| | | Proliferating cell | | | | | |
| | | (Ki88) | | | | | |

TABLE 3
ANTIBODY STAINING RESULTS OBTAINED WITH A SIMMERING TIME OF 20 MINS.

| S | | ANTIBODY | | | PREVIOUS | RESULTS | RESULTS |
|---|---|---|---|---|---|---|---|
| # | ANTI # | NAME | LOT. | TISSUE | PROTOCOL | SLIDE 1 | SLIDE 2 |
| 1 | AM256 | Androgen | 2561095 | Prostate CA | Citra 30 m, RT | 4 | 4 |
| | | receptor | | | | | |
| 2 | AM328 | Progesterone | 3280898C | Breast CA | Citra 30 m, RT | 4.5 | 4.5 |
| | | receptor | | | | | |
| 3 | AM370 | Ki-67 antigen, | 370039S | Tonsil | Citra 2 h, 370 C. | 4.5 | 4.5 |
| | | Proliferating cell | | | | | |
| | | (Ki88) | | | | | |

### Example 10

### Operation of the Microwave Chamber Sealing Means and Real-Time Microwave Processor

The following protocols describe the operation of the microwave chamber sealing means and the real-time microwave processor by a programmable computer and accompanying software in a preferred embodiment of the invention:

Door Open and Close Operations

The door open and close operations are controlled by the hardware and software. The computer sends a signal to turn on the DC motor to drive the door, which slides in a slot on top of the microwave via a lead screw. A sensor detects the door open and closed status via a flag attached to the door. When the computer receives the signal from the sensor the DC motor is stopped. The DC power source is inverted to perform the open and close operations.

Real-time Microwave Processing

Open the door by computer issuing a command from the Galil motion controller (call “CB4”); when the flag reaches the sensor a signal is sent out that the door is opened completely. When the open signal is received, the motion controller issues a command (“SB4”) and the door is held at open position. The Galil motion controller then sends pulses to X, Y, Z, stepping motors to move the slide carrier into the microwave and start fluid filling operation of the tanks. The door is then closed by issuing a close command (CB3) to multifunction board (with inverted DC power to the DC motor). When the flag reaches the close sensor, a signal (SB3) is sent out to stop the motor and turn on the magnetron. The fluid is heated up to the prescribed temperature. When the temperature is reached the thermal sensor sends out a signal to the computer and the heating is stopped. After the prescribed duration the door is opened as described above and the slide carriers are removed by the specimen positioning device.

Although the invention has been described in some detail with reference to the preferred embodiments, those of skill in the art will realize, in light of the teachings herein, that certain changes and modifications can be made without departing from the spirit and scope of the invention. Accordingly, the invention is limited only by the claims.

What is claimed is:

1. An apparatus for automated specimen processing, comprising:
a supporting framework;
a microwave unit attached to the framework comprising a sealable microwave chamber, a microwave source for producing microwaves within the chamber, and power supply means for providing power to the microwave source;
microwave chamber sealing means attached to the microwave chamber;
microwave source control means electrically connected to the microwave source;
at least one microwave tank located within the microwave chamber;
a specimen positioning device attached to the framework that transports the specimen into and out of the microwave chamber; and
specimen positioning device control means electronically connected to the specimen positioning device.

2. The apparatus of claim 1, wherein the microwave source control means comprises a real-time microwave processor.

3. The apparatus of claim 2, wherein the microwave source control means comprises a temperature sensor for measuring the temperature of the solution in one of the microwave tanks.

4. The apparatus of claim 3, wherein the microwave chamber further comprises an inner base wherein a plurality of microwave tanks are positioned.

5. The apparatus of claim 3, wherein the microwave source control means further comprises means to adjustably control the solution temperature of each of the microwave tanks located within the microwave chamber.

6. The apparatus of claim 5, wherein the means to adjustably control the solution temperature of the microwave tanks comprises means to control power to the microwave source by thermocouple and set point.

7. The apparatus of claim 5, wherein the means to adjustably control the solution temperature of the microwave tanks comprises means to control a baffle in the microwave chamber by thermocouple and set point.

8. The apparatus of claim 1, wherein the specimen positioning device comprises a holding element, a three-axis step-motor-driven positioning element that positions the holding element, and holding element control means.

9. The apparatus of claim 1, wherein the specimen positioning device control means comprises a computer-operated motion controller.

10. The apparatus of claim 1, further comprising a specimen identification device attached to the specimen positioning device.

11. The apparatus of claim 10, wherein the specimen identification device comprises a barcode reading scanner.

12. The apparatus of claim 1, further comprising a fluid delivery system comprising a conduit having a first end for attachment to an outlet on a fluid source and a second end directing output of the conduit into a tank attached to the framework directly or indirectly and located inside or outside of the microwave chamber, and fluid delivery control means for regulating output from the conduit.

13. The apparatus of claim 12, wherein the fluid delivery control means comprises a programmable computer system controlling an electrically-operated valve that opens and closes the conduit.

14. The apparatus of claim 12, wherein the fluid delivery system comprises a plurality of conduits each having a first end for attachment to an outlet on a plurality of fluid sources, comprising means for fluid level detection, and each conduit having fluid delivery control means for regulating the delivery of fluid from a second end of each conduit into a tank attached to the framework.

15. The apparatus of claim 12, further comprising a plurality of microwave tanks, wherein the fluid delivery system comprises a plurality of conduits that are joined at first ends to form a manifold and that have a plurality of second ends each directing output into a different tank attached to the framework.

16. The apparatus of claim 1, further comprising an outflow system on the microwave tank, the outflow system comprising an outflow valve, a drainage pump, an outflow valve control means, and at least one waste reservoir comprising means for fluid level detection.

17. The apparatus of claim 1, further comprising at least one staining tank attached to the framework outside of the microwave chamber.

18. The apparatus of claim 1, further comprising at least one rinse tank attached to the framework outside of the microwave chamber.

19. The apparatus of claim 18, further comprising means for fluid level detection in each rinse tank.

20. The apparatus of claim 19, wherein the means for fluid level detection uses pressure sensor means.

21. The apparatus of claim 19, wherein the means for fluid level detection uses optical sensor means.

22. The apparatus of claim 19, wherein the means for fluid level detection uses electrical sensor means.

23. The apparatus of claim 1, further comprising at least one holding tank attached to the framework outside of the microwave chamber for storing the specimen in a solution.

24. The apparatus of claim 23, further comprising means for fluid level detection in each holding tank.

25. The apparatus of claim 24, wherein the means for fluid level detection uses pressure sensor means.

26. The apparatus of claim 24, wherein the means for fluid level detection uses optical sensor means.

27. The apparatus of claim 24, wherein the means for fluid level detection uses electrical sensor means.

28. The apparatus of claim 1, further comprising means for fluid level detection in each microwave tank.

29. The apparatus of claim 28, wherein the means for fluid level detection uses pressure sensor means.

30. The apparatus of claim 28, wherein the means for fluid level detection uses optical sensor means.

31. The apparatus of claim 28, wherein the means for fluid level detection uses electrical sensor means.

32. The apparatus of claim 1, further comprising means for exchanging the air in the microwave chamber.

33. The apparatus of claim 32, wherein means for exchanging the atmosphere in the microwave chamber comprises an internal exhaust fan and at least one atmospheric filter.

34. The apparatus of claim 1, wherein the apparatus can operate on either 110 VAC or 220 VAC.

35. An apparatus for automated specimen processing, comprising:
a supporting framework;
a microwave unit attached to the framework comprising a sealable microwave chamber attached to the framework, a microwave source attached to and directing microwaves into the chamber, and power supply means electrically connected to the microwave source;
microwave chamber sealing means attached to the chamber;
an interlock sensor electrically connected to the microwave source that delivers a signal when the microwave chamber is not sealed which prevents the production of microwaves by the microwave source;
microwave source control means electrically connected to the microwave source comprising a real-time microwave processor, a temperature sensor for measuring the temperature of the solution in the microwave tank, and a programmable computer system;
at least one microwave tank located within the microwave chamber, wherein each microwave tank has means for fluid level detection and an outflow system leading out of the chamber for removing a solution from the microwave tank, the outflow system comprising an outflow valve, a drainage pump and outflow valve control means;
means for controlling microwave tank solution temperature;
a specimen positioning device attached to the framework comprising a holding element, a three-axis step-motor-driven positioning element that positions the holding element, and a computer-operated motion controller that controls the positioning element and the holding element;
a barcode reading scanner attached to the specimen positioning device and electrically connected to a programmable computer system;
a loading area located attached to the framework outside of the microwave chamber for retaining the specimen prior to heating;
a rinse tank attached to the framework outside of the microwave chamber comprising means for fluid level detection;
at least one holding tank attached to the framework outside of the microwave chamber for storing the specimen, and comprising means for fluid level detection; and
a fluid delivery system comprising a conduit having a first end for attachment to an outlet on a fluid source, comprising means for fluid level detection, and a second end directing output of the conduit into the microwave tank, fluid delivery control means regulating output from the conduit, and at least one waste reservoir comprising means for fluid level detection.

36. An automated method for dewaxing a specimen, comprising:
transporting the specimen into a microwave tank in a microwave chamber using a computer-controlled specimen positioning device;
providing a dewaxing solution within the tank; and
heating the dewaxing solution and specimen using a computer-controlled microwave unit so that the solution maintains a temperature of from about 60° C. to just below the solution boiling point for at least about 3 minutes.

37. The method of claim 36, further comprising replacing the dewaxing solution with fresh dewaxing solution and heating the fresh dewaxing solution and specimen using the computer-controlled microwave unit so that the fresh dewaxing solution maintains a temperature of from about 60° C. to just below the solution boiling point for at least about 3 minutes.

38. An automated method of hematoxylin and eosin staining, comprising:
transporting a specimen into a hematoxylin tank using a computer-controlled specimen positioning device;
providing a solution of hematoxylin in the hematoxylin tank;
contacting the specimen with the hematoxylin solution for a time sufficient to stain the specimen with sufficient hematoxylin to be detected by light microscopy;
transporting the specimen into a rinse tank;
providing rinse solution in the rinse tank;
contacting the specimen with the rinse solution for a plurality of times sufficient to minimize specimen background and maximize the useful lifetime of staining solutions;
transporting the specimen into an eosin tank using a computer-controlled specimen positioning device;
providing a solution of eosin in the eosin tank;
contacting the specimen with the solution of eosin for a time sufficient to stain the specimen with sufficient eosin to be detected by light microscopy; and
removing the specimen from the solution of eosin using a computer-controlled specimen positioning device.

39. The method of claim 38, further comprising:
transporting the specimen into a rinse tank;
providing rinse solution in the rinse tank;
contacting the specimen with the rinse solution for a plurality of times sufficient to minimize specimen background and maximize the useful lifetime of differentiating solutions;
transporting the specimen into a differentiation tank using a computer-controlled specimen positioning device;
providing a differentiation solution in the differentiation tank; and
contacting the specimen with the differentiation solution and then with a clearing solution to prepare for permanent mounting.

40. An automated method of antigen retrieval or nucleic acid retrieval, comprising:
transporting a specimen into a microwave tank in a microwave chamber using a computer-controlled specimen positioning device;
providing a solution within the tank;
heating the solution and specimen in the tank using a computer-controlled microwave unit so that the solution maintains a temperature of from about 50° C. to just below the solution boiling point for a period of time sufficient to enhance immunostaining of the specimen; and
allowing the heated solution containing the specimen to gradually cool.

41. The method of claim 40, further comprising unsealing the microwave chamber.

42. The method of claim 40, wherein the solution is chelating agents, citrate salts, metal ions, Citra, Citra Plus, AR-10, or Glyca.

43. The method of claim 36, wherein the apparatus first moves a specimen identification device attached to the specimen positioning device to at least one work area of the apparatus selected from the group consisting of a loading area, the microwave tank, a staining tank, a rinse tank and a holding tank to determine if a specimen is already present in the work area.

44. The method of claim 38, wherein the apparatus first moves a specimen identification device attached to the specimen positioning device to at least one work area of the apparatus selected from the group consisting of a loading area, a microwave tank, the eosin tank, the hematoxylin tank, a differentiation tank, a staining tank, a rinse tank and a holding tank to determine if a specimen is already present in the work area.

45. The method of claim 40, wherein the apparatus first moves a specimen identification device attached to the specimen positioning device to at least one work area of the apparatus selected from the group consisting of a loading area, the microwave tank, a staining tank, a rinse tank and a holding tank to determine if a specimen is already present in the work area.

46. A process for tracking a plurality of specimens, comprising identifying each of the specimens using a specimen identification device attached to a three-axis computer-controlled specimen positioning device, assigning a unique location within a computer memory system to each specimen using a computer program, and updating the location of each specimen as the computer-controlled specimen positioning device moves each specimen to different locations in the apparatus while performing an automated histotechnological method.

47. The apparatus of claim 1, wherein the microwave tank accommodates slide racks of a plurality of sizes and/or shapes.

48. The apparatus of claim 35, wherein the loading area accommodates slide racks of a plurality of sizes and/or shapes.

49. A microscope slide comprising an identification label on an edge of the slide.

50. The microscope slide of claim 49, wherein the identification label is optically detectable.

51. The microscope slide of claim 49, wherein the identification label is a barcode.

52. An automated method for dewaxing and antigen retrieval of a specimen, comprising:
transporting the specimen into a microwave tank in a microwave chamber using a computer-controlled specimen positioning device;
providing a dewaxing solution within the tank;
heating the dewaxing solution and specimen using a computer-controlled microwave unit so that the solution maintains a temperature of from about 60° C. to just below the solution boiling point for at least about 3 minutes to about 10 minutes;
replacing the dewaxing solution with a fresh solution within the tank;
heating the solution and specimen in the tank using a computer-controlled microwave unit so that the solution maintains a temperature of from about 50° C. to just below the solution boiling point for a period of time sufficient to enhance immunostaining of the specimen; and
allowing the heated solution containing the specimen to gradually cool.

53. The method of claim 52, further comprising unsealing the microwave chamber.

54. The method of claim 52, wherein the fresh solution is chelating agents, citrate salts, metal ions, Citra, Citra Plus, AR-10, or Glyca.

55. The apparatus of claim 35, wherein the specimen positioning device comprises means for sensing slide racks.
